SEQUENCE LISTING (27 sequences)

SEQ ID NO: 1 - DNA, 24 bp, synthetic construct
  source 1..24: mol_type = other DNA
  misc_feature 1..24: note = Gene: CCNA2, Function: Target, Oligo: Forward Primer
tgctgttagcctcaaagtttgaag

SEQ ID NO: 2 - DNA, 25 bp, synthetic construct
  source 1..25: mol_type = other DNA
  misc_feature 1..25: note = Gene: CCNA2, Function: Target, Oligo: Reverse Primer
cttggtgtaggtatcatctgtaatg

SEQ ID NO: 3 - DNA, 29 bp, synthetic construct
  source 1..29: mol_type = other DNA
  misc_feature 1..29: note = Gene: CCNA2, Function: Target, Oligo: Probe
tataccccccagaagtagcagagtttgtg

SEQ ID NO: 4 - DNA, 23 bp, synthetic construct
  source 1..23: mol_type = other DNA
  misc_feature 1..23: note = Gene: PCNA, Function: Target, Oligo: Forward Primer
cctgaacttctttacaaaagcca

SEQ ID NO: 5 - DNA, 22 bp, synthetic construct
  source 1..22: mol_type = other DNA
  misc_feature 1..22: note = Gene: PCNA, Function: Target, Oligo: Reverse Primer
ctctacaacaaggggtacatct

SEQ ID NO: 6 - DNA, 26 bp, synthetic construct
  source 1..26: mol_type = other DNA
  misc_feature 1..26: note = Gene: PCNA, Function: Target, Oligo: Probe
ctcttcaacggtgacactcagtatgt

SEQ ID NO: 7 - DNA, 20 bp, synthetic construct
  source 1..20: mol_type = other DNA
  misc_feature 1..20: note = Gene: KIF23, Function: Target, Oligo: Forward Primer
atcattgcggcaggttactc

SEQ ID NO: 8 - DNA, 18 bp, synthetic construct
  source 1..18: mol_type = other DNA
  misc_feature 1..18: note = Gene: KIF23, Function: Target, Oligo: Reverse Primer
gcagagcgtgatcgtctg

SEQ ID NO: 9 - DNA, 25 bp, synthetic construct
  source 1..25: mol_type = other DNA
  misc_feature 1..25: note = Gene: KIF23, Function: Target, Oligo: Probe
caacctgatcagaacgcaccaccaa

SEQ ID NO: 10 - DNA, 20 bp, synthetic construct
  source 1..20: mol_type = other DNA
  misc_feature 1..20: note = Gene: MCM2, Function: Target, Oligo: Forward Primer
cgcttcaagaacttcctgcg

SEQ ID NO: 11 - DNA, 21 bp, synthetic construct
  source 1..21: mol_type = other DNA
  misc_feature 1..21: note = Gene: MCM2, Function: Target, Oligo: Reverse Primer
ctcacggttctctttgcacat

SEQ ID NO: 12 - DNA, 25 bp, synthetic construct
  source 1..25: mol_type = other DNA
  misc_feature 1..25: note = Gene: MCM2, Function: Target, Oligo: Probe
ccacaacgtcttcaaggagcgcatc

SEQ ID NO: 13 - DNA, 20 bp, synthetic construct
  source 1..20: mol_type = other DNA
  misc_feature 1..20: note = Gene: MCM4, Function: Target, Oligo: Forward Primer
aaggactacattgcctacgc

SEQ ID NO: 14 - DNA, 23 bp, synthetic construct
  source 1..23: mol_type = other DNA
  misc_feature 1..23: note = Gene: MCM4, Function: Target, Oligo: Reverse Primer
catgtctacataagcctcgatga

SEQ ID NO: 15 - DNA, 21 bp, synthetic construct
  misc_feature 2..21: note = DNA
  source 1..21: mol_type = other DNA
  misc_feature 1: note = RNA: Locked nucleic acid (LNA)
  misc_feature 1..21: note = Gene: MCM4, Function: Target, Oligo: Probe
cggctaagtgaggaagccagc

SEQ ID NO: 16 - DNA, 24 bp, synthetic construct
  source 1..24: mol_type = other DNA
  misc_feature 1..24: note = Gene: AURKA, Function: Target, Oligo: Forward Primer
ccaccttcggcatcctaatattct

SEQ ID NO: 17 - DNA, 23 bp, synthetic construct
  source 1..23: mol_type = other DNA
  misc_feature 1..23: note = Gene: AURKA, Function: Target, Oligo: Reverse Primer
tgttccaagtggtgcatattcca

SEQ ID NO: 18 - DNA, 25 bp, synthetic construct
  misc_feature 1..2: note = DNA
  misc_feature 3: note = RNA: Locked nucleic acid (LNA)
  misc_feature 4..16: note = DNA
  source 1..25: mol_type = other DNA
  misc_feature 1..25: note = Gene: AURKA, Function: Target, Oligo: Probe
  misc_feature 17: note = RNA: Locked nucleic acid (LNA)
  misc_feature 18..25: note = DNA
ttccatgatgctaccagagtctacc

SEQ ID NO: 19 - DNA, 22 bp, synthetic construct
  source 1..22: mol_type = other DNA
  misc_feature 1..22: note = Gene: MKI67, Function: Target, Oligo: Forward Primer
gcttgtttggaaggggtattga

SEQ ID NO: 20 - DNA, 23 bp, synthetic construct
  source 1..23: mol_type = other DNA
  misc_feature 1..23: note = Gene: MKI67, Function: Target, Oligo: Reverse Primer
ctgctcatggatttcaattttgc

SEQ ID NO: 21 - DNA, 24 bp, synthetic construct
  source 1..24: mol_type = other DNA
  misc_feature 1..24: note = Gene: MKI67, Function: Target, Oligo: Probe
agcttcctgttgtgtcaaaacaac

SEQ ID NO: 22 - DNA, 21 bp, synthetic construct
  source 1..21: mol_type = other DNA
  misc_feature 1..21: note = Gene: IPO8, Function: Ref Gene, Oligo: Forward Primer
catcattcttcagtgcaaagg

SEQ ID NO: 23 - DNA, 21 bp, synthetic construct
  source 1..21: mol_type = other DNA
  misc_feature 1..21: note = Gene: IPO8, Function: Ref Gene, Oligo: Reverse Primer
ctctccaaaacaagttgaacg

SEQ ID NO: 24 - DNA, 28 bp, synthetic construct
  source 1..28: mol_type = other DNA
  misc_feature 1..28: note = Gene: IPO8, Function: Ref Gene, Oligo: Probe
aggggaattgatcagtgcattccactct

SEQ ID NO: 25 - DNA, 22 bp, synthetic construct
  source 1..22: mol_type = other DNA
  misc_feature 1..22: note = Gene: PUM1, Function: Ref Gene, Oligo: Forward Primer
catccttgggattcggaagtag

SEQ ID NO: 26 - DNA, 21 bp, synthetic construct
  source 1..21: mol_type = other DNA
  misc_feature 1..21: note = Gene: PUM1, Function: Ref Gene, Oligo: Reverse Primer
cagtgttggagtttgcaactg

SEQ ID NO: 27 - DNA, 20 bp, synthetic construct
  source 1..20: mol_type = other DNA
  misc_feature 1..20: note = Gene: PUM1, Function: Ref Gene, Oligo: Probe
cccttggagggtttggaaca

(19) United States
(12) Patent Application Publication
(10) Pub. No.: US 2025/0320561 A1
(43) Pub. Date: Oct. 16, 2025

(54) DIAGNOSTIC TEST

(71) Applicant: Apis Assay Technologies Ltd., Manchester (GB)

(72) Inventors: Joanna Gorniak, Manchester (GB); Sara Rollinson, Manchester (GB)

(21) Appl. No.: 18/714,670

(22) PCT Filed: Dec. 1, 2022
(86) PCT No.: PCT/GB2022/053048

§ 371 Date: May 30, 2024

(30) Foreign Application Priority Data
Dec. 2, 2021 (GB) 2117404.0

(51) Int. Cl. C12Q 1/6886; C12Q 1/6851

(52) U.S. Cl. CPC C12Q 1/6886; C12Q 1/6851; C12Q 2600/118; C12Q 2600/158

(57) ABSTRACT

The present invention relates to methods for classifying a patient's cancer, including determining the proliferation status of the cancer and for classifying the cancer. Such methods also allow for prognosing or predicting clinical outcome of a patient with cancer; for selecting a treatment regime for a patient with cancer; and, to treatments directed to a patient whose cancer has been classified using the methods of the invention. The present invention also provides kits for use in the methods of the invention.

### RELATED APPLICATIONS

This application is a § 371 national-stage application based on PCT/GB22/53048, filed Dec. 1, 2022; which claims the benefit of priority to U.K. Patent Application No. 2117404.0, filed Dec. 2, 2021.

### FIELD

The present invention relates to methods for classifying a patient's cancer, including determining the proliferation status of the cancer and for classifying the cancer. Suitably, the cancer is breast cancer and the method allows the breast cancer to be classified as being luminal A or luminal B subtype. Such methods also allow for prognosing or predicting clinical outcome of a patient with cancer; for selecting a treatment regime for a patient with cancer; and, to treatments directed to a patient whose cancer has been classified using the methods of the invention, suitably the cancer is breast cancer. The present invention also provides kits for use in the methods of the invention.

### BACKGROUND

Breast cancer (BC) is the most common cancer reported in women, with an estimated 2.1 million new cases and 627,000 deaths in 2018 (Bray et al., CA Cancer J Clin 68 (6): 394-424, 2018). Using gene expression profiling Perou and Sorlie demonstrated that breast tumours could be categorised by their “intrinsic” molecular subtype (Ozmen, V., Journal of Breast Health (13): 50-53, 2017).

As defined by the St. Gallen guidelines (Cardoso, F. et al., Annals of Oncology (30): 1194-1220, 2019) the disease can be segregated into five main molecular subtypes: Luminal A-like; Luminal B-like HER2-negative; Luminal B-like HER2-positive; Non-luminal Her2-positive; and Triple negative. The subtypes are determined by combining the expression status of four markers, the hormone receptors for Oestrogen and Progesterone (ER and PR), Human Epidermal Growth Factor Receptor 2 (Her2), and the proliferation marker KI67 (Goldhirsch, A. et al., Annals of Oncology (24): 2206-2223, 2013). The molecular subtype can be reconstructed by using immunohistochemistry (IHC) which measures the protein expression of the markers. The marker status and subtype in breast cancer can be used to predict a response to therapy and is used to inform treatment decisions (Table 1).

TABLE 1
Breast cancer molecular subtypes, marker profile and associated
adjuvant therapies adapted from Eliyatkin et al. (Eliyatkin,
N., Yalcin, E., Zengel, B., Akta [symbol] , S. & Vardar,
E. Molecular Classification of Breast Carcinoma: From Traditional,
Old-Fashioned Way to A New Age, and A New Way. J. Breast
Heal. (2015). doi: 10.5152/tjbh.2015.1669).

| Subtype | Surrogate IHC Markers | Note/therapy |
|---|---|---|
| Luminal A | Positive ER and or PR | Endocrine therapy |
| | Negative HER2, | Good prognosis |
| | Low KI67 | |
| Luminal B | Positive ER and or PR | Endocrine therapy plus |
| (HER2 | Negative HER2 | chemotherapy Prognosis not |
| negative) | High KI67 | as good as luminal A |
| Luminal B | High ER and or PR | Endocrine therapy/chemotherapy |
| (HER2 | HER2 positive | plus ERBB2 targeted therapy |
| positive) | High KI67 | Usually, unfavourable prognosis |
| HER2 | Negative ER and PR | Endocrine therapy/chemotherapy |
| positive | HER2 positive | with anthracyclines plus HER2 |
| | High KI67 | targeted therapy |
| Triple | Negative ER, PR | Chemotherapy with platinum group |
| Negative | and HER2 High KI67 | agents and PARP inhibitors |
| | | Usually, worse prognosis |
| Basal | Negative ER, PR and | Chemotherapy with platinum group |
| | HER2 High KI67, | agents and PARP inhibitors |
| | positive CK5 | Usually, worse prognosis |

Hormone receptor positivity (ER and PR) in breast cancers predicts a potential response to endocrine-based treatments, positive status being associated with a good prognosis. HER2 positive tumours are treated with anti-HER2 therapy and chemotherapy. Tumours are also characterised by their proliferative fraction (most commonly assessed by KI67 immunostaining) (Cardoso, F. et al., Annals of Oncology (30): 1194-1220, 2019). High proliferation predicts chemosensitivity and along with grade is a major factor in the recommendation of chemotherapy (Harbeck, N. et al., Breast cancer. Nat. Rev. Dis. Prim 23; 5 (1): 66, 2019) for hormone receptor positive tumours. Patients may also be offered additional treatments, such as alternative targeted therapies, or chemotherapy, depending upon other clinical features, such as tumour size or lymph node involvement, and risk of relapse.

Evaluation of ER and PR is a standard practise and most often performed by IHC. Up to 80% of breast cancer cases are ER positive, and 55-65% positive for PR (Waks A G et al, JAMA; (321): 288-300, 2019). The American Society of Clinical Oncology (ASCO) and College of American Pathologists (CAP) provide recommendations for ER/PR measurement and reporting (Alfarsi, et al., Histopathology (73): 545-58, 2018). Central review from clinical trials report up to 21.4% false negatives for ER (Van Bockstal, et al., Breast (Edinburgh, Scotland) (37) 52-5, 2018). Retesting of hormone receptors centrally result in high levels of reproducibility however demonstrating the intra lab reproducibility issues that occur with IHC (Bartlett, J. et al., Journal of the National Cancer Institute; 108 2016, Hall P S et al, Value in health: the journal of the International Society for Pharmacoeconomics and Outcomes Research; 20:1311-8, 2017). HER2 expression is initially assessed by IHC, a score of 2+ is accorded to samples when there is either incomplete or weak to moderate circumferential membrane staining in >10% of invasive tumour cells. Scores of 2+ are referred to fluorescence in situ hybridisation (FISH) to determine HER2 amplification (Lv, Q. et al., International Journal of Molecular Sciences (17): 2095, 2016). Central review in clinical trials reports up to 14.5% false positive results for HER2 (Van Bockstal et al, Breast (Edinburgh, Scotland) (37) 52-5, 2018). The introduction of standardized methodologies for testing ER, PR and HER2 along with participation in external testing programs are going some way to improve the lack of reproducibility seen with IHC between testing sites (Van Bockstal, et al., Breast (Edinburgh, Scotland) (37) 52-5, 2018). KI67 IHC staining is also commonly performed, however whilst intra-laboratory reproducibility of Ki-67 IHC staining is high, substantial inter-laboratory variability is observed even when the same antibody is used (Focke, C. M. et al., Eur. J. Cancer (84): 219-227, 2017). The measurement of proliferation in breast cancer is not optimized. RT-qPCR based in vitro diagnostic assays, including the MammaTyper and Xpert® Breast Cancer STRAT4 show low concordance with IHC Ki-67 results. However, gene expression measurement of MKI67 show promise in improving diagnostic accuracy. (Sinn, H. P. et al. BMC Cancer, 17 (1): 124, 2017., Sinn, P. et al., Geburtshilfe und Frauenheilkunde 73 (9): 932-940, 2013., Varga, Z. et al., Breast Cancer Res 19 (1): 55, 2017).

Minimizing the use of chemotherapy for breast cancer treatment is of key importance in standard of care and patient quality of life, however identifying patients who will obtain maximal benefit from chemotherapy is a non-trivial task. At present tools derived from clinical features combined with ER/PR/HER2 status are used as a guide to determine the prognosis of the patient, such as the Nottingham Prognostic Index (NPI; Todd et al., Br J Cancer 56 (4): 489-92, 1987) or PREDICT (Candido Dos Reis et al., Breast Cancer Res. 22; 19 (1): 58, 2017).

For hormone receptor positive (ER/PR), Her2-negative patients the decision to administer chemotherapy can be unclear. For patients with an intermediate risk of recurrence e.g. an intermediate NPI score (3.4<NPI≤5.4), the cost of treatment and side effects to the patients could outweigh the potential survival benefit of chemotherapy. In these cases, molecular in vitro diagnostic (IVD) assays, including Oncotype Dx™, Prosigna™, and EndoPredict™ may be employed. These tests determine the expression levels of multiple genes producing a risk score which defines the risk of relapse. These tests can be time consuming, costly and show low concordance in patients with intermediate risk (Sestak et al., JAMA Oncol. 4 (4): 545-553, 2018).

One of the principal abnormalities resulting in the development of cancer is the unregulated proliferation of cancer cells. Tumour cells do not respond to cellular checkpoints, and grow and divide in an uncontrolled manner, if unchecked eventually spreading throughout the body. As such the proliferative activity of tumour cells represents an important prognostic marker in the diagnosis of cancer. For diagnostic purposes the most widely used marker of proliferation is KI67, in addition to breast cancer the marker is utilised for the differential diagnosis in lymphoma (Jain and Wang., AJH. 94 (6): 710-725, 2019. Higgins et al., Archives of Pathology & Laboratory Medicine. 132 (3): 441-461, 2008), adreno cortico cancers (Vargas, et al, Am J Surg Pathol. 21 (5): 556-62, 1997), and cervical carcinomas/adenocarcinomas (Carreras et al, Histol Histopathol. 22 (6): 587-92, 2007; Wu et al, Ann Palliat Med. 10 (9): 9544-9552, 2021). High KI67 expression has been associated with poor prognosis in Ovarian (Grabowski et al, Int J Gynecol Cancer. 30 (4): 498-503, 2020) adreno cortico cancers (Vargas, et al, Am J Surg Pathol. 21 (5): 556-62, 1997), lung (Grant et al, Horm Cancer. 9 (4): 288-294, 2018), mantle cell lymphoma (Jain and Wang., AJH. 94 (6): 710-725, 2019), Bladder (Amin et al, Am J Surg Pathol. 38 (8):e20-34) and urothelial carcinomas (Mallofre et al, Mod Pathol. 16 (3): 187-91, 2003). Proliferation measurement is being used to predict prognosis in Mantle cell lymphoma and response to R-CHOP therapeutic regimes (Scott et al, J Clin Oncol. 20; 35 (15): 1668-1677, 2017). The accurate measurement of proliferation opens up many potential benefits for the diagnosis and treatment of these and other tumour types.

### SUMMARY OF THE INVENTION

The present invention is based in part on the identification of biomarkers whose expression levels, individually or in combination, can be used to determine the proliferation status of the cancer cells in a patient with cancer. The proliferation status can be determined for any type of cancer but breast cancer is of particular relevance. Suitably, aspects of the invention can be used to classify the cancer as luminal A or luminal B subtype. In addition, the expression levels of the biomarkers can be used for prediction or prognosis purposes, such as their likelihood of responding to a particular treatment or their long-term clinical outcome (such as progression free survival, overall survival or likelihood of relapse).

According to a first aspect of the invention there is provided a method for classifying a patient's cancer comprising determining the expression level of at least one biomarker selected from: KIF23, AURKA, MCM2, CCNA2, PCNA and MCM4 and classifying the breast cancer based on the expression levels of the biomarker detected. In a particular embodiment, the classification is determining the proliferation status of the cancer. In a particular embodiment, the cancer is breast cancer.

Suitably, the method involves determining the expression level of at least two biomarkers selected from: MKI67, KIF23, AURKA, MCM2, CCNA2, PCNA and MCM4, optionally wherein one of the at least two biomarkers is MKI67.

Suitably, the method involves determining the expression level of at least four biomarkers including: MKI67, KIF23, CCNA2 and PCNA.

The method is carried out on a suitable biological sample from the patient (i.e. in vitro). Typically, the sample has been previously isolated from the patient, such as via biopsy.

The expression levels of the biomarker(s) can be used to determine the proliferation status of the cancer, for example to classify the patient's cancer as being of high or low proliferation status. Additionally, the expression levels of the biomarker(s) can be used to classify a breast cancer as being luminal A or luminal B subtype.

The ability to classify the patient's cancer according to their proliferation status, or to classify a breast cancer as being luminal A or luminal B subtype offers up the ability to use these biomarkers and the method of the first aspect of the invention for predictive and/or prognostic purposes. For example, to predict likelihood of a patient to respond to a particular treatment, and thus to select a suitable treatment regime for the patient. Alternatively, to prognose the likely development of the disease and clinical outcome, including the ability to prognose likelihood of long-term survival (such as 2-, 5- or 10-year survival). The method according to the first aspect of the invention can therefore be used as a tool in the clinical management of a patient's cancer.

According to a second aspect of the invention there is provided a method for prognosing or predicting clinical outcome for a patient with cancer, comprising determining the expression level of at least one biomarker selected from: KIF23, AURKA, MCM2, CCNA2, PCNA and MCM4, and prognosing or predicting the clinical outcome for the cancer patient from these expression values. Suitably, the method involves determining the expression level of at least two biomarkers selected from: MKI67, KIF23, AURKA, MCM2, CCNA2, PCNA and MCM4, optionally wherein one of the at least two biomarkers is MKI67. Suitably, the method involves determining the expression level of a panel of biomarkers comprising at least: MKI67, KIF23, CCNA2 and PCNA. Suitably the panel comprises between 4 and 8 biomarkers. Suitably the cancer is breast cancer.

Suitably the expression level of the biomarker(s) can be determined by detecting the amount of protein or the amount of RNA transcript that encodes the biomarker present in the patient sample. Typically, these expression levels are normalised against suitable reference genes/proteins, as appropriate.

In particular embodiments, the methods of the invention can be carried out alongside other methods, such as established methods, for classifying or subtyping the cancer. For example, for breast cancer the methods of the invention can be carried out in conjunction with methods that employ determining the expression level of one, some or all of the following biomarkers: ESR1, PGR, ERBB2 and keratin 5.

By “in conjunction with” we mean that the methods of the invention can be carried out separately but likely in parallel with methods that determine the expression levels of one or more other markers. By way of example, with breast cancer the methods of the invention can be carried out separately but likely in parallel with methods that determine the expression levels of one or more of ESR1, PGR, ERBB2 and keratin 5 (for example as separate tests), or the detection of the biomarkers used in the methods of the present invention and one or more additional biomarkers (such as one or more of ESR1, PGR, ERBB2 and keratin 5 for breast cancer) can be carried out together, for example as part of the same test.

The incorporation of detection of the expression level of one or more of the biomarkers used in the present invention with the expression level of one or more of ESR1, PGR, ERBB2 and keratin 5 will allow a patient's breast cancer to be classified as luminal A-like, luminal B-like (HER2 negative), luminal B-like (HER2 positive), HER2 positive (non-luminal) or Triple Negative.

The ability to classify or sub-type the patient's cancer is fundamental to determining the appropriate treatment regime and gaining knowledge of the disease and likely prognosis for the patient.

According to a third aspect of the invention there is provided a treatment recommendation or guiding treatment decisions for a patient with hormone receptor positive HER2 negative breast cancer, comprising determining the expression levels of a panel of biomarkers, applying these to an algorithm capable of identifying whether the patient is likely to have a luminal A or luminal B cancer and making a treatment recommendation based thereon, wherein the panel of biomarkers comprises (i) at least one of: KIF23, AURKA, MCM2, CCNA2, PCNA or MCM4; or (ii) MKI67 and at least one of the following: KIF23, AURKA, MCM2, CCNA2, PCNA or MCM4.

The methods of the present invention allow to determine whether the patient has luminal A or luminal B type breast cancer, particularly for patients whose cancer has already been determined to be hormone positive (e.g. ER and/or PR positive) and Her2 negative. The luminal A or B characterisation reflects the type of disease and thus can influence or determine the appropriate treatment regime for the patient.

According to a fourth aspect of the invention there is provided a method of treating a patient with breast cancer, comprising determining whether the patient has luminal A or luminal B breast cancer according to the method of the first aspect of the invention, wherein if the patient's cancer is classified as luminal A they are treated, with bisphosphonates and/or endocrine therapy, and if the patient's cancer is classified as luminal B they are treated with a regime that includes bisphosphonates, endocrine therapy and/or chemotherapy. Suitably, the breast cancer is hormone receptor positive Her2 negative.

According to a variant of the fourth aspect of the invention there is provided a drug selected from tamoxifen, raloxifene, fulvestrant, toremifene, goserelin, leuprolide, triptorelin, anastrozole, exemestane, and letrozole, palbociclib, ribociclib, abemaciclib, an anthracycline, e.g. doxorubicin hydrochloride or epirubicin hydrochloride; a taxane, e.g. paclitaxel or docetaxel; a vinca alkaloid, e.g. eribulin mesylate or vinblastine; an antimetabolite, e.g. capecitabine, fluorouracil, gemcitabine hydrochloride or methotrexate; a platinum-based agent, e.g. carboplatin or cisplatin; an epothilone, e.g. ixabepilone; cyclophosphamide; a Cdk4/6 inhibitor, e.g. abemaciclib, palbociclib or ribociclib; a poly-ADP-ribose polymerase (PARP) inhibitor, e.g. olaparib or talazoparib; a PI3K inhibitor, e.g. alpesilib; a PD-L1 inhibitor, e.g. atezolizumab or pembrolizumab; an aurora kinase inhibitor, e.g. alisertib, an ubiquitin proteasome inhibitor, a BCL2-inhibitor, an mTOR inhibitor, e.g. everolimus, a bisphosphonate agent, e.g. zoledronic acid or sodium clodronate, for use in a method of treating luminal B breast cancer, wherein the method comprises determining whether patient cancer has luminal A or luminal B subtype breast cancer according to the method of the first aspect of the invention, and if the patient has luminal B breast cancer, administering an effective amount of the drug to the patient.

According to a variant of the fourth aspect of the invention there is provided a drug selected from tamoxifen, raloxifene, fulvestrant, toremifene, goserelin, leuprolide, triptorelin, anastrozole, exemestane, and letrozole, palbociclib, ribociclib, and abemaciclib for use in a method of treating luminal A breast cancer, wherein the method comprises determining whether patient cancer has luminal A or luminal B subtype breast cancer according to the method of the first aspect of the invention, and if the patient has luminal A breast cancer, administering an effective amount of the drug to the patient.

The present invention also provides a kit comprising one or more reagents suitable for determining the expression levels of the biomarkers measured in the methods of the invention described herein.

According to fifth aspect of the invention there is provided a kit of parts comprising a set of oligonucleotide primer pairs wherein each primer pair is capable of selectively hybridising to one of the transcripts in a panel of genes and creating a PCR amplification product, and instructions for use, wherein the panel of genes comprises at least 2 genes selected from the group consisting of: KIF23, CCNA2, MKI67, AURKA, MCM2, MCM4 and PCNA. Optionally, the kit also comprises at least one probe capable of selectively hybridising to each amplification product. Optionally, the kit also comprises at least one set of primers capable of selectively hybridising to one of the transcripts selected from the group consisting of: ESR1, PGR, ERBB2 and keratin 5, and creating a PCR amplification product, and optionally at least one probe capable of selectively hybridising to the amplification product. Optionally the probe is labelled, such as with a fluorescent label, to aid detection. Optionally, the kit also comprised a pair of amplification primers and an amplification product detection probe for a reference gene. Optionally, the kit also comprises instructions for use.

Suitably, the kit also comprises means for interpreting the expression data, such means could be a software package capable of weighting the expression levels of each detected biomarker and providing a call (such as: yes/no, high/low proliferation score, or luminal A/luminal B).

According to a sixth aspect of the invention there is provided a kit of parts comprising at least one binding moiety capable of specifically binding to a biomarker protein selected from: KIF23, CCNA2, AURKA, MCM2, MCM4 and PCNA. Suitably the binding moiety is an antibody or antibody fragment capable of selectively binding to the biomarker. Optionally, the binding moiety is labelled, such as fluorescently labelled. Optionally, the kit also comprised a binding moiety for a reference protein.

Optionally, the kit also comprises instructions for use. Suitably, the kit also comprises means for interpreting the expression data, such means could be a software package capable of weighting the expression levels of each detected biomarker and providing a call (such as: yes/no, high/low proliferation score, or luminal A/luminal B).

According to a seventh aspect the invention there is provided a biomarker selected from: KIF23, AURKA, MCM2, CCNA2, PCNA and MCM4 for use as a cancer subtyping marker or as a marker of relapse free survival. Suitably, the cancer is breast cancer.

As will be appreciated by the person of skill in the art, the third, fourth, fifth, sixth and seventh aspects of the invention can use or incorporate the method of the first or second aspects of the invention.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following:

### DETAILED DESCRIPTION

The disclosed methods may be understood more readily by reference to the following detailed description which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

The methods of the present disclosure will employ, unless otherwise indicated, conventional techniques of molecular biology (including recombinant techniques), microbiology, cell biology, and biochemistry, which are within the skill of the art. Exemplary techniques are explained fully in the literature, such as, “Molecular Cloning: A Laboratory Manual”, 2nd edition (Sambrook et al., Molecular Cloning: A Laboratory Manual, New York: Cold Spring Harbor Press, 1989); “Current Protocols in Molecular Biology” (F. M. Ausubel et al., eds., Current Protocols of Molecular Biology, John Wiley and Sons (1987); and “PCR: The Polymerase Chain Reaction”, (Mullis et al., eds., Birhäuser, Boston, 1994).

Unless indicated otherwise, each gene name used herein corresponds to the Official Symbol assigned to the gene and provided by Entrez Gene (URL: www.ncbi.nlm.nih.gov/sites/entrez) as of the filing date of this application.

### Definitions

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed methods, which are for clarity, described herein in the context of separate embodiments, may also be provided in combination in a single embodiment. Conversely, various features of the disclosed methods that are, for brevity, described in the context of a single embodiments, may also be provided separately or in any sub-combination.

The articles “a,” “an,” and “the” are used herein to refer to one or to more than one (i.e. to at least one) of the grammatical object of the article.

The use of the alternative (e.g., “or”) should be understood to mean either one, both, or any combination thereof of the alternatives.

The term “and/or” should be understood to mean either one, or both of the alternatives.

When values are expressed as approximations, by use of the antecedent “about”, it will be understood that the particular value forms another embodiment.

As used herein and unless stated otherwise, it is to be understood that the term “about” is used synonymously with the term “approximately”. Illustratively and unless stated otherwise, the use of the term “about” indicates values slightly outside the cited criteria values, for example ±15%, ±10%±8%, ±5% or conveniently ±2%. Such values are thus encompassed by the scope of the claims reciting the terms “about” or approximately”.

The term “biomarker” in the context of the present invention encompasses, without limitation, a gene (e.g. nucleic acid), including messenger RNA, and its encoded protein or polypeptide.

Table 2 identifies the main biomarkers disclosed herein, including their gene identifier and Ensemble ID which provides details of the gene and protein sequences.

TABLE 2
Biomarker information

| | | Entrez | |
|---|---|---|---|
| Gene | | Gene | |
| Symbol | Gene Name | ID | Ensembl ID |
| ESR1 | Oestrogen Receptor 1 | 2099 | ENSG00000091831 |
| PGR | Progesterone Receptor | 5241 | ENSG00000082175 |
| ERBB2 | Erb-b2 receptor tyrosine | 2064 | ENSG00000141736 |
| | kinase 2 | | |
| KI67 | Marker of proliferation Ki-67 | 4288 | ENSG00000148773 |
| KRT5 | Keratin 5 | 3852 | ENSG00000186081 |
| KIF23 | Kinesin family member 23 | 9493 | ENSG00000137807 |
| AURKA | Aurora kinase A | 6790 | ENSG00000087586 |
| MCM2 | Minichromosome | 4171 | ENSG00000073111 |
| | maintenance | | |
| | complex component 2 | | |
| MCM4 | Minichromosome | 4173 | ENSG00000104738 |
| | maintenance | | |
| | complex component 4 | | |
| CCNA2 | Cyclin A2 | 890 | ENSG00000145386 |
| PCNA | Proliferating cell nuclear | 5111 | ENSG00000132646 |
| | antigen | | |

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene.

The term “subsequence” refers to a sequence of nucleic acids that comprises a part of a longer nucleic acid sequence. An exemplary subsequence is a probe, described herein, or a primer. The term “primer” as used herein refers to a contiguous sequence comprising for example, about 8 or more deoxyribonucleotides or ribonucleotides, such as 10-20, 15-25 or 20-30 nucleotides of a selected nucleic acid molecule. The primers of the presently disclosed subject matter encompass oligonucleotides of sufficient length and appropriate sequence so as to provide initiation of polymerization on a nucleic acid molecule of the presently disclosed subject matter.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein.

The term “amplify” is used in the broad sense to mean creating an amplification product, which for example, can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

Nucleic acid amplification can be carried out by numerous methods generally known to the person skilled in the art such as, but not limited to, polymerase chain reaction (PCR), isothermal amplification, ligation amplification (or ligase chain reaction, LCR), real time (rtPCR), quantitative PCR (qPCR), digital droplet PCR (ddPCR), or probe based PCR methods, and other amplification methods. These methods are generally known. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR).

By “homologous” is meant that a nucleic acid molecule shares a substantial amount of sequence identity with another nucleic acid molecule. Substantial amount means at least 90%, such as at least 95%, at least 98% and more usually at least 99%. Suitably, sequence identity is determined using the BLAST algorithm, as described in Altschul et al. (1990), J. Mol. Biol. 215:403-410 (e.g. using the published default setting, i.e. parameters w=4, t=17).

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules (such as in a patient's sample). A “target sequence” is synonymous with a “test sequence”. Suitably, a probe for detection of the presence of a target can be labelled.

The term “label” refers to a composition capable of producing a detectable signal indicative of the presence of the labelled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, for example at least about 50/cm2, at least about 100/cm2, at least about 500/cm2, but preferably below about 1,000/cm2. In certain embodiments, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

“Expression” and “gene expression” include transcription and/or translation of nucleic acid material.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays can be coupled with processes or systems to store and process information and to help quantify levels, signals, etc. and to digitize the information for use in comparing levels

As used herein, the term “in vitro” means performed or taking place in a test tube, culture dish, or elsewhere outside a living organism. The term also includes ex vivo because the analysis takes place outside an organism.

As used herein, the term “isolated” means material that is substantially or essentially free from components that normally accompany it in its native state. In the context of isolated from a subject it can mean is removed from the subject. In particular embodiments, the term “obtained” or “derived” is used synonymously with isolated.

A “subject,” “individual,” or “patient” as used herein, includes any animal that can be tested using the present invention. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in a particular embodiment, the subject is a human.

As used herein, “prognosis” is a forecast of the course of a disease following its onset and typically refers to the possible outcomes of a disease (e.g. death, chance of recovery, recurrence).

Whilst prognosis and prediction are sometimes used interchangeably, the term “prediction” herein, refers to an assessment of the likelihood of responding favourably to a treatment.

Unless defined otherwise all technical and scientific terms used herein have the same meaning as commonly understood to one of ordinary skill in the art to which this invention belongs.

According to a first aspect of the invention there is provided a method for classifying a patient's cancer comprising determining the expression level of at least one biomarker selected from: KIF23, AURKA, MCM2, CCNA2, PCNA and MCM4, and based on the expression levels of the biomarker detected determining the proliferation status of the cancer.

Suitably, the method involves determining the expression level of at least two biomarkers selected from: MKI67, KIF23, AURKA, MCM2, CCNA2, PCNA and MCM4, optionally wherein one of the at least two biomarkers is MKI67.

Suitably, the method involves determining the expression level of at least four biomarkers including MKI67, KIF23, CCNA2 and PCNA.

In a particular embodiment, the proliferation status is used to determine a suitable treatment option for the patient.

The method is carried out on a suitable biological sample from the patient. Typically, the sample has been previously isolated from the patient, such as via biopsy.

In a particular embodiment, the cancer is breast cancer.

The expression levels of the biomarker(s) can be used to determine the proliferation status of the cancer, for example to classify a patient's cancer as being of high or low proliferation status.

The expression levels of the biomarker(s) can be used to classify a breast cancer as being luminal A or luminal B subtype.

In a particular embodiment, the method has a performance of discriminating between luminal A or luminal B (Her2 negative) subtype of at least 0.7 F1 and/or an accuracy of at least 0.8 and/or a precision of at least 0.75.

The ability to classify the patient's cancer according to their proliferation status, or, with respect to a breast cancer as being luminal A or luminal B subtype, offers up the ability to use these biomarkers and the method of the first aspect of the invention for predictive and/or prognostic purposes. For example, to predict likelihood of a patient to respond to a particular treatment, and thus to select a suitable treatment regime for the patient. Alternatively, to prognose the likely development of the disease and clinical outcome, including the ability to predict or prognose likelihood of long-term survival (such as 2-, 5- or 10-year survival) or likelihood of cancer recurrence (e.g. within a period of time, such as within 2, 5, 7 or 10 years). The method according to the first aspect of the invention can therefore be used as a tool in the clinical management of the patient's cancer.

According to a second aspect of the invention there is provided a method for prognosing or predicting clinical outcome for a patient diagnosed with cancer, comprising determining the expression level of at least one biomarker selected from: KIF23, AURKA, MCM2, CCNA2, PCNA and MCM4, and prognosing or predicting the clinical outcome for the cancer patient from these expression values. Alternatively, the method involves determining the expression level of at least two biomarkers selected from: MKI67, KIF23, AURKA, MCM2, CCNA2, PCNA and MCM4, optionally wherein one of the at least two biomarkers is MKI67. Suitably, the cancer is breast cancer.

In particular embodiments, the expression level of each biomarker is weighted to determine the cancer proliferation status and/or classify a breast cancer as being a luminal A or luminal B subtype.

In particular embodiments, the expression level of at least 3, 4, 5, 6, or all 7, biomarkers are determined.

In particular embodiments, the expression level of a panel of biomarkers comprising MKI67, KIF23, CCNA2 and PCNA are determined.

In a particular embodiment, the method of the first or second aspect of the invention can be used to classify whether the patient's breast cancer is luminal A or luminal B subtype.

In particular embodiments, the expression levels detected are compared to or correlated with reference values characteristic of luminal A and luminal B breast cancer subtypes.

The method is particularly suited to subtyping patients whose cancer is known to be hormone receptor positive (e.g. ER and/or R positive) and Her2 negative.

In a particular embodiment, the patient's cancer is hormone receptor positive and Her2 negative. Suitably, the hormone receptor positive and Her2 negative status has already been determined prior to conducting the method of the first or second aspect of the invention.

In particular embodiments, the prognosis measure is a period of survival, such as 2, 3, 5, 7, 10 years or more survival. Suitably, the time period will run from an accepted clinical determination, such as from original cancer diagnosis or from the time the sample analysed by the methods of the invention is taken.

In a particular embodiment, the prognosis measure is determining the likelihood of cancer recurrence.

In a particular embodiment, there is a provided a method for determining the likelihood of cancer recurrence in a human subject comprising:
(a) determining the normalised expression levels of at least 2 biomarkers selected from the group consisting of: MKI67, KIF23, AURKA, MCM2, CCNA2, PCNA and MCM4, optionally wherein at least one of the biomarkers is MKI67, in a biological sample comprising breast cancer cells obtained from the subject;
(b) weighting the normalised expression levels of each biomarker; and
(c) determining the likelihood of cancer recurrence based on the data in step (b).

Suitably, with this embodiment the cancer is breast cancer.

In particular embodiments, the biomarker expression levels are transformed into a clinical outcome forecast by an algorithm.

The methods of the first and second aspects of the invention are carried out on a suitable biological sample from the patient. The sample typically comprises cancer cells, e.g. tumour biopsies or blood samples that contain circulating cancer (tumour) cells (CTCs), or comprises nucleic acid derived from cancer cells, e.g. circulating free nucleic acid in blood or a nucleic acid containing sample that has been amplified from an original patient sample. Typically, the sample has been previously isolated from the patient, such as via biopsy.

In particular embodiments, the sample comprises a biofluid or bodily fluid. In some embodiments, the biofluid or bodily fluid is obtainable from the subject by way of non-invasive or minimally invasive methods.

In particular embodiments, the sample comprises a tissue sample.

In particular embodiments, the diagnostic methods of the invention are carried out on fresh samples, frozen samples or formalin-fixed paraffin-embedded tissue samples.

The biomarker expression level in a sample can be obtained through quantifying the expression levels of mRNA or protein corresponding to one or more of the biomarkers identified herein.

Expression data may be generated by one or more of a range of gene or protein expression measuring techniques, for example but not limited to, RT-PCR, Affymetrix whole genome microarray profiling for gene expression or immunohistochemistry (IHC) or Western Blotting for protein expression. Normalisation is typically applied to the raw data generated from these techniques to remove any artefactual inter-subject differences arising from sample processing, sample quality or sample quantity. Data from RT-PCR is typically normalised by subtracting the average expression of one or more normalisation or reference genes from the observed expression levels of the gene of interest for each sample. Data from Affymetrix whole genome microarrays is typically normalised by the RMA algorithm or one of a number of alternative algorithms including but not limited to MAS5, PLIER, GC-RMA.

A method (or assay) of the invention may utilize a means related to the expression level of a biomarker disclosed herein as long as the assay reflects, quantitatively or qualitatively, expression of the biomarker. In one embodiment a quantitative assay is performed. The ability to discriminate is conferred by the identification of expression of the individual biomarkers as relevant and not by the form of the assay used to determine the actual level of expression. An assay may utilize any identifying feature of an identified individual biomarker as disclosed herein as long as the assay reflects, quantitatively or qualitatively, expression of the biomarker. Identifying features include, but are not limited to, unique nucleic acid sequences used to encode (DNA), or express (RNA), said gene or epitopes specific to, or activities of, a protein encoded by said gene. Alternative means include detection of nucleic acid amplification as indicative of increased expression levels and nucleic acid inactivation, deletion, or methylation, as indicative of decreased expression levels. The invention may be practiced by assaying one or more aspect of the DNA template(s) underlying the expression of the disclosed sequence(s), or the RNA used as an intermediate to express the sequence(s), or of the proteinaceous product expressed by the sequence(s), as well as proteolytic fragments of such products. As such, the detection of the presence of, amount of, stability of, or degradation (including rate) of, such DNA, RNA and proteinaceous molecules may be used in the practice of the invention. Accordingly, all that is required is an appropriate cancer cell-containing sample from the patient for analysis.

In particular embodiments, the expression level for each measured biomarker is determined quantitatively.

In this context, “quantitively” also covers semi-quantitatively and is a measurement of gene expression quantity.

According to particular embodiments, the expression level of each biomarker is normalised, such as by use of one or more reference genes or other reference measurements, such as a reference protein measure.

To determine the expression levels of the biomarkers in the practice of the present invention, any method known in the art may be utilized. In one embodiment of the invention, expression based on detection of mRNA is used.

The various aspects of the invention are suitable for use with any cancer, including liquid cancers (including haematological cancers such as leukaemia) and solid tumours (such as lymphoma, renal, lung, colon, prostate, ovarian, skin, bladder, urothlial and cervical cancer).

The various aspects of the invention are particularly suited for use with breast cancer.

### RNA Detection

In a particular embodiment, the biomarker expression level for each biomarker is determined based on the amount of the RNA transcript level detected.

In a particular embodiment, the expression level for each biomarker is determined as part of reverse transcriptase polymerase chain reaction (RT-PCR). Suitably, the RT-PCR is quantitative reverse-transcription polymerase chain reaction (RT-qPCR). In a particular embodiment, the RT-qPCR is carried out on total RNA extracted from one or more slices or sections of the breast cancer sample. In other embodiments, the RT-qPCR is carried out using primers capable of selectively hybridising to the target gene transcripts in the panel of genes.

In a particular embodiment, the RNA transcript expression level of the or each biomarker is measured using RT-PCR and the, or each, expression level is normalised, optionally to get delta CT.

One embodiment of the invention involves determining expression by hybridization of mRNA, or an amplified or cloned version thereof, to a polynucleotide that is unique to a particular biomarker gene sequence. In one embodiment, one or more sequences capable of hybridising to one or more of the genes identified herein is immobilised on a solid support or microarray.

Alternatively, solution-based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes (up to one nucleotide shorter than the full length sequence known in the art by deletion from the 5′ or 3′ end of the sequence) that are optionally minimally interrupted (such as by mismatches or inserted non-complementary base pairs) such that hybridization with a DNA or RNA corresponding to the gene(s) is not affected. In certain embodiments, the polynucleotides used are from the 3′ end of the gene, such as within about 350, about 300, about 250, about 200, about 150, about 100, or about 50 nucleotides from the polyadenylation signal or polyadenylation site of a gene or expressed sequence.

Preferred polynucleotides contain at least about 18, at least about 20, at least about 22, at least about 24, at least about 26, at least about 28, at least about 30, or at least about 32, at least about 34, at least about 36, at least about 38, at least about 40, at least about 42, at least about 44, or at least about 46 consecutive base pairs of a gene sequence that is not found in other gene sequences. The term “about” as used in the previous sentence refers to an increase or decrease of 1 from the stated numerical value. Even more preferred are polynucleotides of at least or about 50, at least or about 100, at least about or 150, at least or about 200, at least or about 250, at least or about 300, at least or about 350, or at least or about 400 base pairs of a gene sequence that is not found in other gene sequences. The term “about” as used in the preceding sentence refers to an increase or decrease of 10% from the stated numerical value. Such polynucleotides may also be referred to as polynucleotide probes that are capable of hybridizing to sequences of the genes, or unique portions thereof, described herein. In one embodiment, the sequences are those of mRNA encoded by the genes, the corresponding cDNA to such mRNAs, and/or amplified versions of such sequences. In certain embodiments of the invention, the polynucleotide probes are immobilized on a microarray, other devices, or in individual spots that localize the probes on a support. Polynucleotides containing mutations relative to the sequences of the disclosed genes may also be used so long as the presence of the mutations still allows hybridization to produce a detectable signal.

Suitably, all or part of a disclosed biomarker sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a disclosed sequence, where the primers are used to prime nucleic acid synthesis. The newly synthesized nucleic acids are optionally labelled and may be detected directly or by hybridization to a polynucleotide of the invention. The newly synthesized nucleic acids may be contacted with biomarker polynucleotides of the invention under conditions, which allow for their hybridization. Additional methods to detect the expression of expressed nucleic acids include RNAse protection assays, including liquid phase hybridizations, and in situ hybridization of cells. The Ct values generated by such methods may be used to generate the ratios of expression levels as described herein.

In particular embodiments, in the method of the invention, the expression level is determined by reverse transcription polymerase chain reaction (RT-PCR).

In some embodiments, the expression level of the RNA transcript can be determined by nucleic acid sequencing. Sequencing can be carried out by any method known in the art including, but not limited to, sequencing by hybridization, sequencing by ligation or sequencing by synthesis. Sequencing by ligation includes, but is not limited to, fluorescent in situ sequencing (FISSEQ). Sequencing by synthesis includes, but is not limited to, reversible terminator chemistry (i.e. Illumina SBS).

A quantitative sequencing-based approach such as serial analysis of gene expression (SAGE) (see Velculescu et al. Science. 20:270 (5235): 484-487, 1995) (including variants thereof) or RNA-Sequencing (RNA-Seq) can also be used. RNA-Seq refers to the use of any of a variety of high throughput sequencing techniques to quantify RNA transcripts (see, e.g., Wang et al. Nature Reviews Genetics. 10:57-63, 2009). Other methods of use for detecting RNA include, e.g., electrochemical detection, bioluminescence-based methods, fluorescence-correlation spectroscopy, etc. It will be understood that certain methods that detect mRNA may, in some instances, also detect at least some pre-mRNA transcript(s), transcript processing intermediates, and degradation products of sufficient size. It will also be understood that a probe or primer may in some embodiments be substantially or perfectly complementary to a complement of the subunit of the PBAF complex RNA.

In embodiments where only one or a few genes are to be analysed, the nucleic acid derived from the sample cancer cell(s) may be preferentially amplified by use of appropriate primers such that only the genes to be analysed are amplified to reduce contaminating background signals from other genes expressed in the cancer cell. Alternatively, and where multiple genes are to be analysed or where very few cells (or one cell) are used, the nucleic acid from the sample may be globally amplified before hybridization to the immobilized polynucleotides. Of course, RNA or the cDNA counterpart thereof may be directly labelled and used, without amplification, by methods known in the art.

The amplified product can be detected using a suitable hybridisation probe using any of a variety of techniques at the disposal of those skilled in the art. Suitably, the hybridisation probe will be capable of hybridising specifically (also referred to as selectively) to the target nucleic acid, which could be a nucleic acid amplification product. Suitably, the probe will be labelled, for example, radioactively, fluorescently or enzymatically. A nucleic acid probe can be of any length, such as at least 15, 20, 25, 30, 40, 50, 60, 70, 80, 90, 100, 150, 200 bases in length, but is typically the same length or shorter than the target nucleic acid, and capable of specifically hybridising to the target sequence. Examples of probe lengths include 10-30, 15-50, 30-70 bases in length.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

Those skilled in the art are able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42° C. in 6×SSC and then washing in 6×SSC at a series of increasing temperatures from 42° C. to 65° C. For example, probes may be washed in 6×SSC at 42° C. for 30 minutes then 6×SSC at 50° C. for 45 mins then 2×SSC for 45 mins at 65° C. Other suitable conditions and protocols are described in Molecular Cloning: a Laboratory Manual: 3rd edition, Sambrook & Russell (2001) Cold Spring Harbor Laboratory Press NY and Current Protocols in Molecular Biology, Ausubel et al. eds. John Wiley & Sons (1992).

### Protein Detection

Alternatively, and in yet another embodiment of the invention, biomarker expression may be determined at the protein level, e.g. by measuring the levels of polypeptides encoded by the biomarker gene products described herein, or activities thereof. Such methods are well known in the art and include, e.g., any immunohistochemistry (IHC) based, antibody (including autoantibodies against the protein) based, mass spectroscopy based, and image (including used of labelled ligand) based method known in the art and recognized as appropriate for the detection of the protein.

In a particular embodiment, the biomarker expression level for each biomarker is determined based on the amount of the biomarker protein detected.

In one embodiment the detection is via an immunoassay that uses one or more antibodies specific for one or more epitopes of the biomarker protein in a cell sample of interest. Any biological material can be used for the detection/quantification of the biomarker protein.

The biomarker proteins can be detected in any suitable manner but are typically detected by contacting a sample from the patient with an antibody that binds the biomarker protein and then detecting the presence or absence of a reaction product. Such as, by use of labelled antibodies against cell surface markers followed by fluorescence activated cell sorting (FACS). Such antibodies are preferably labelled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

Antibodies that can be used herein may be monoclonal, polyclonal, chimeric, or a fragment of the foregoing, as discussed elsewhere herein, preferably monoclonal antibodies, and the step of detecting the reaction product may be carried out with any suitable immunoassay. Antibodies can be commonly used in the art, such as fusion methods (Kohler and Milstein, European Journal of Immunology, 6:511-519 (1976)), recombinant DNA methods (US Pat. No. 4,816,56) or phage antibody library methods (Clackson et al, Nature, 352:624-628 (1991) and Marks et al, J. Mol. Biol., 222:58, 1-597 (1991)). General procedures for antibody preparation are described in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Florida, 1984; And Coligan, CURRENT PROTOCOLS IN IMMUNOLOGY, Wiley/Greene, NY, 1991, which are incorporated herein by reference.

An antibody is optionally conjugated with a detectable label. An intact antibody, a fragment thereof (e.g., Fab or F(ab′) 2), or an engineered variant thereof (e.g., sFv) can also be used. Such antibodies can be of any immunoglobulin class including IgG, IgM, IgE, IgA, IgD and any subclass thereof.

Techniques for detecting antibody binding through the use of a detectable label are well known in the art. For example, antibody binding may be detected through the use of chemical reagents that generate a detectable signal that corresponds to the level of antibody binding and, accordingly, to the level of biomarker protein expression. In some embodiments, the detection antibody is coupled to an enzyme, particularly an enzyme that catalyses the deposition of a chromogen at the antigen-antibody binding site. Suitable enzymes include but are not limited to horseradish peroxidase (HRP) and alkaline phosphatase (AP). Commercial antibody detection systems may also be used to practice the invention.

Although antibodies are illustrated herein for use in the invention because of their extensive characterization, any other suitable agent (e.g., a peptide, an aptamer, or a small organic molecule) that specifically binds a biomarker is optionally used in place of the antibody. For example, an aptamer that specifically binds a selected biomarker may be used. Aptamers are nucleic acid-based molecules that bind specific ligands. Methods for making aptamers with a particular binding specificity are known in the art.

The sample from the subject is typically a solid tissue sample, e.g. a biopsy, as described above, but may be a cancer cell containing biological fluid, e.g. blood or serum sample. The sample may be in the form of a tissue specimen from a patient where the specimen is suitable for immunohistochemistry in a variety of formats such as paraffin-embedded tissue, frozen sections of tissue, and freshly isolated tissue. The immunodetection methods are antibody-based but there are numerous additional techniques that allow for highly sensitive determinations of binding to an antibody in the context of a tissue. Those skilled in the art will be familiar with various immunohistochemistry strategies.

Immunoassays carried out in accordance with the present invention may be homogeneous assays or heterogeneous assays. In a homogeneous assay the immunological reaction usually involves the specific antibody (e.g., anti-biomarker protein antibody), a labelled analyte, and the sample of interest. The signal arising from the label is modified, directly or indirectly, upon the binding of the antibody to the labelled analyte. Both the immunological reaction and detection of the extent thereof are carried out in a homogeneous solution. Immunochemical labels that may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels.

For example, if the protein (or polypeptide) to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are radioimmunoassays, immunofluorescence methods, chemiluminescence methods, electrochemiluminescence or enzyme-linked immunoassays.

Those skilled in the art will be familiar with numerous specific immunoassay formats and variations thereof, which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,1 10 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies may be conjugated to a solid support suitable for a diagnostic assay (e.g., beads, plates, slides or wells formed from materials such as latex or polystyrene) in accordance with known techniques, such as passive binding.

Antibodies as described herein may likewise be conjugated to detectable groups such as radiolabels (e.g., 35S, 1251, 1311), enzyme labels (e.g., horseradish peroxidase, alkaline phosphatase), and fluorescent labels (e.g., fluorescein) in accordance with known techniques.

Alternative methods of detecting a protein biomarker in a sample include high performance liquid chromatography (HPLC) and other high-throughput techniques.

Additionally, identification and quantification of one or more biomarkers can be performed using mass spectrometry. One specific example of mass spectrometry that may be useful is tandem mass spectrometry; another example is high mass accuracy/high mass resolution mass spectrometry (e.g. Orbitrap™, Thermo Scientific).

Tandem mass spectrometry, for example, can be used for quantitative analysis of peptides in biological samples due to high sensitivity and specificity. High mass accuracy/high mass resolution mass spectrometers (e.g. Orbitrap™, Thermo Scientific) also can be utilized for analysis. Generally, a product of digestion can be purified using separation techniques and ionized to generate ions detectable by mass spectrometry, where the concentration of peptides is determined by mass spectrometry, and amount detected is related to the amount of biomarker in the test sample. The ions can be single charged or multiple charged. In one aspect, ions selected in the first stage of mass analysis can be monoisotopic or isotopic. In another aspect, ions selected in the second stage of mass analysis can be monoisotopic or isotopic. Additionally, it is contemplated that in some cases ions selected in all following stages of mass analysis can be monoisotopic or isotopic.

Measurements can be obtained separately for individual parameters or can be obtained simultaneously for a plurality of parameters. Any suitable platform can be used to obtain parameter measurements.

The skilled artisan can routinely make antibodies, nucleic acid probes, e.g., oligonucleotides, aptamers, siRNAs against any of the biomarkers identified herein.

While even a single biomarker can be used to determine the proliferation status and/or luminal A/B subtype status of a patient's cancer, the present invention may be practised with two or more of the biomarkers in combination to increase the accuracy of the method.

### Data Analysis

In certain embodiments the expression level of the biomarker(s) can be compared to that detected in control cell(s), which may be obtained from non-cancerous tissue from the same or a different individual. Suitable controls include non-cancer cells from the same tissue or lineage. Comparison can be performed on test and reference samples measured concurrently or at temporally distinct times. An example of the latter is the use of compiled expression information, e.g., a sequence database, which assembles information about expression levels of the biomarker(s). In some embodiments, the pattern of biomarker expression in the test sample is measured and then may be normalised against one or more control or reference genes. Examples of reference genes against which the biomarker expression levels can be normalised include but are not limited to: PUM1 and/or IPO8.

Thus, according to particular embodiments of the first or second aspects of the invention, expression level of each biomarker is normalised, such as by reference to the expression level of at least one reference gene. Suitably, the at least one reference gene comprises PUM1 and/or IPO8.

Increases or decreases in expression of the biomarkers disclosed herein can be determined based upon percent or fold changes over expression in normal cells, reference cells or normalised against one or more reference or housekeeping genes/biomarkers. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10-fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells. For example, a 2-fold increase or decrease is a useful measure for determining whether or not the expression level is low or high. The threshold level of expression of any biomarker used according to the invention can be determined empirically using clinical samples and an appropriate algorithm as described further herein.

A mathematical algorithm which has been pre-determined by the analysis of preclinical or historical clinical data can then be applied to the expression measures to give a forecast or call. The algorithm may apply a weighting to the expression level of each or any biomarker.

Suitably, the algorithm has been generated by machine learning trained on archived samples from patients whose (i) luminal A and/or luminal B (Her2 negative) status is known; or (ii) cancer recurrence outcome is known. Suitably, the weighting applied to the expression level of each biomarker has been generated by this machine learning approach.

A number of different statistical algorithms can be applied in order to generate a mathematical model combining together the expression levels of two or more genes or proteins with a cut-off to classify the patient's cancer. These include, but are not limited to logistic regression, multiple regression, Cox proportional hazard models, random forests, recursive partitioning, random survival forests, partial least squares, partial least squares discriminant analysis, Support Vector Machines, neural networks.

### Multiple Biomarker Assessment

The biomarkers identified herein may be used singly with significant accuracy or in combination to increase the ability to accurately determine proliferation status and/or luminal A/B subtype.

In particular embodiments of the first or second aspects of the invention, the method comprises determining the expression level MKI67 and at least one biomarker, such as 1, 2, 3, 4, 5 or 6 biomarkers selected from: KIF23, CCNA2, AURKA, MCM2, MCM4 and PCNA.

In particular embodiments, the method according to the first or second aspects of the invention, comprises determination of the expression levels of at least two biomarkers selected from: KIF23, CCNA2, MKI67, AURKA, MCM2, MCM4 and PCNA.

In particular embodiments of the first or second aspects of the invention, the expression level of at least 3, 4, 5, 6, or all 7, biomarkers are determined.

In particular embodiments, the method according to the first or second aspects of the invention, comprises determination of the expression levels of at least three biomarkers selected from: KIF23, CCNA2, MKI67, AURKA, MCM2, MCM4 and PCNA.

In particular embodiments, the method according to the first or second aspects of the invention, comprises determination of the expression levels of at least four biomarkers selected from: KIF23, CCNA2, MKI67, AURKA, MCM2, MCM4 and PCNA. Suitably, the method according to the first or second aspects of the invention, comprises determination of the expression levels a panel of biomarkers comprising: MKI67, KIF23, CCNA2 and PCNA, and optionally one or more biomarkers.

In particular embodiments, the method according to the first or second aspects of the invention, comprises determination of the expression levels of at least five biomarkers selected from: KIF23, CCNA2, MKI67, AURKA, MCM2, MCM4 and PCNA.

In particular embodiments, the method according to the first or second aspects of the invention, comprises determination of the expression levels of at least six biomarkers selected from: KIF23, CCNA2, MKI67, AURKA, MCM2, MCM4 and PCNA.

In particular embodiments, the method according to the first or second aspects of the invention, comprises determination of the expression levels of each of the following biomarkers: KIF23, CCNA2, MKI67, AURKA, MCM2, MCM4 and PCNA.

In particular embodiments of the first or second aspects of the invention the biomarkers evaluated comprises:
at least four biomarkers selected from: KIF23, CCNA2, MKI67, AURKA, MCM2, MCM4 and PCNA; or
MKI67 and at least three biomarkers selected from KIF23, CCNA2, AURKA, MCM2, MCM4 and PCNA; or
MKI67, KIF23, CCNA2 and PCNA.

In other embodiments the biomarkers evaluated comprise each biomarker in a group selected from the following groups (i)-(xx):

| | |
|---|---|
| (i) | Kif23, CCNA2, AURKA, MKI67; |
| (ii) | MCM2, CCNA2, AURKA, MKI67; |
| (iii) | MCM2, Kif23, AURKA, MKI67; |
| (iv) | MCM2, Kif23, CCNA2, MKI67; |
| (v) | MCM2, MCM4, AURKA, MKI67; |
| (vi) | MCM2, MCM4, CCNA2, MKI67; |
| (vii) | MCM2, MCM4, Kif23, MKI67; |
| (viii) | MCM4, CCNA2, AURKA, MKI67; |
| (ix) | MCM4, Kif23, AURKA, MKI67; |
| (x) | MCM4, Kif23, CCNA2, MKI67; |
| (xi) | PCNA, CCNA2, AURKA, MKI67; |
| (xii) | PCNA, Kif23, AURKA, MKI67; |
| (xiii) | PCNA, Kif23, CCNA2, MKI67; |
| (xiv) | PCNA, MCM2, AURKA, MKI67; |
| (xv) | PCNA, MCM2, CCNA2, MKI67; |
| (xvi) | PCNA, MCM2, Kif23, MKI67; |
| (xvii) | PCNA, MCM2, MCM4, MKI67; |
| (xviii) | PCNA, MCM4, AURKA, MKI67; |
| (xix) | PCNA, MCM4, CCNA2, MKI67; and |
| (xx) | PCNA, MCM4, Kif23, MKI67. |

The methods of the invention are suitable for classifying a cancer as having high or low proliferation status. The methods of the invention are suitable for classifying a breast cancer as being luminal A or luminal B subtype (particularly if the cancer is hormone receptor positive/Her2 negative). Such methods can therefore be included with or combined with other known cancer classification methods or assays.

Currently, breast cancer sub-typing can be carried out using an in vitro diagnostic test intended to determine the relative expression of five mRNA target genes-ESR, PGR, ERBB2, MKI67 and KRT5. The test is designed to report the individual biomarker status (Positive or Negative) alongside the molecular classification as described in the St Gallen (2017) guidelines (Cardoso, F. et al., Annals of Oncology (30): 1194-1220, 2019). Molecular classifications are described as luminal A-like, luminal B-like (HER2 negative), luminal B-like (HER2 positive), HER2 positive (non-luminal) and Triple Negative.

As an alternative to mRNA level determination, immunohistochemistry (IHC) can be used to determine the status of ER, PR, HER2, KI67 and CK5. The marker status and subtype in breast cancer can be used to predict a response to therapy, and as such inform treatment decisions (Table 1). However, as explained above, significant challenges and deficiencies exist in correlating the RT-PCR and IHC detection of MKI67 using the current test.

The present invention provides an alternative to the use of MKI67 alone as the sole biomarker for determining proliferation status and breast cancer sub-type.

In particular embodiments, the methods of the invention can be carried out alongside other methods, such as established methods, for classifying or subtyping a cancer, such as breast cancer. For example, for breast cancer, in conjunction with methods that employ determining the expression level of one, some or all of the following biomarkers: ESR1, PGR, ERBB2 and keratin 5.

Thus, in particular embodiments of any of aspects 1, 2 or 3 of the invention, the method for classifying a patient's breast cancer also comprises determining the expression level of at least one of the following biomarkers: ESR1, PGR, ERBB2 and keratin 5.

Such method can be used to classifying a patient's breast cancer as Luminal A-like, Luminal B-like (HER2 negative), Luminal B-like (HER2 positive), HER2 positive (non-luminal) or Triple Negative.

Thus, according to a further aspect of the invention there is provided a method for classifying a patient's breast cancer as Luminal A-like, Luminal B-like (HER2 negative), Luminal B-like (HER2 positive), HER2 positive (non-luminal) or Triple Negative comprising, determining the expression level of (i) at least one biomarker selected from: KIF23, AURKA, MCM2, CCNA2, PCNA and MCM4; or (ii) at least two biomarkers selected from: MKI67, KIF23, AURKA, MCM2, CCNA2, PCNA and MCM4, are measured, optionally wherein one of the at least two biomarkers is MKI67; and in addition to (i) or (ii), determining the expression level of at least one of the following biomarkers: ESR1, PGR, ERBB2 and keratin 5; and classifying the patient's breast cancer as being Luminal A-like, Luminal B-like (HER2 negative), Luminal B-like (HER2 positive), HER2 positive (non-luminal) or Triple Negative based on the expression levels of the biomarkers determined.

In a particular embodiment, for (i), the expression level of a panel of biomarkers comprising MKI67, KIF23, CCNA2 and PCNA are measured.

In a particular embodiment, the expression level of ESR1 is used to determine if the patient's cancer is hormone receptor positive (Luminal A, Luminal B HER2 negative, or Luminal B HER2 positive).

In a particular embodiment, the expression level of PGR is used to determine if the patient's cancer is (Luminal A, Luminal B HER2 negative, or Luminal B HER2 positive).

In a particular embodiment, the expression level of ERBB2 is used to determine if the patient's cancer is Luminal B HER2 positive or HER2 positive.

In a particular embodiment, the expression level of keratin 5 is used to determine if the patient's cancer is the basal subgroup of the Triple negative subtype.

In particular embodiments of aspects 1 or 2 of the invention, the method for classifying a patient's breast cancer also comprises determining the expression level of each of the following biomarkers: ESR1, PGR, ERBB2 and keratin 5; and wherein the expression levels of the biomarkers facilitate classifying the patient's cancer as Luminal A-like, Luminal B-like (HER2 negative), Luminal B-like (HER2 positive), HER2 positive (non-luminal) or Triple Negative.

The ability to classify or sub-type the patient's breast cancer is fundamental to determining the appropriate treatment regime and gaining knowledge of the disease and likely prognosis for the patient.

### Treatment Applications

The diagnostic methods of the invention permit the determination of the proliferation status of a cancer. Knowing the proliferation status (i.e. how aggressive the cancer is) allows the physician to decide a suitable treatment regime for the patient.

The diagnostic methods of the invention permit the classification of a patient's breast cancer as being luminal A or luminal B subtype, particularly for patients whose cancer has already been determined to be hormone positive (e.g. ER and/or PR positive) and Her2 negative, as well as determine the proliferation status of the cancer. The proliferation status or luminal A or B classification reflects the type of disease and thus can influence or determine the appropriate treatment regime for the patient. Accordingly, the present invention also opens up the possibility of treating the patient based on whether their cancer is determined to be luminal A or B subtype, and/or is a high or low proliferation cancer according to the methods of the first or second aspects of the invention.

The methods of the present invention allow to determine whether the patient has luminal A or luminal B type breast cancer.

According to current clinical guidelines patients with luminal A hormone receptor positive Her2 negative cancers are treated with a drug selected from: tamoxifen, raloxitene, fulvestrant, toremifene, goserelin, leuprolide, triptorelin, anastrozole, exemestane, and letrozole, palbociclib, ribociclib, and abemaciclib.

According to current clinical guidelines patients with luminal B hormone receptor positive Her2 negative cancers are treated with a drug selected from: tamoxifen, raloxifene, fulvestrant, toremifene, goserelin, leuprolide, triptorelin, anastrozole, exemestane, and letrozole, palbociclib, ribociclib, abemaciclib, an anthracycline, e.g. doxorubicin hydrochloride or epirubicin hydrochloride; a taxane, e.g. paclitaxel or docetaxel; a vinca alkaloid, e.g. eribulin mesylate or vinblastine; an antimetabolite, e.g. capecitabine, fluorouracil, gemcitabine hydrochloride or methotrexate; a platinum-based agent, e.g. carboplatin or cisplatin; an epothilone, e.g. ixabepilone; cyclophosphamide; a Cdk4/6 inhibitor, e.g. abemaciclib, palbociclib or ribociclib; a poly-ADP-ribose polymerase (PARP) inhibitor, e.g. olaparib or talazoparib; a PI3K inhibitor, e.g. alpesilib; a PD-L1 inhibitor, e.g. atezolizumab or pembrolizumab; an aurora kinase inhibitor, e.g. alisertib, an ubiquitin proteasome inhibitor, a BCL2-inhibitor, an mTOR inhibitor, e.g. everolimus, an aromatase inhibitor, e.g. letrozole, anastrozole, exemestane, and tamoxifen, a bisphosphonate agent, e.g. zoledronic acid or sodium clodronate.

By way of example, tamoxifen can be administered to men or pre-menopausal women, identified as having luminal A or low proliferation status. Whereas postmenopausal patients identified as having medium to high recurrence risk can be administered an aromatase inhibitor in addition to tamoxifen.

According to a third aspect of the invention there is provided a method for providing a treatment recommendation or guiding treatment decisions for a patient with breast cancer, comprising determining whether the patient's cancer is luminal A or luminal B according to the method of the first or second aspect of the invention, and providing a treatment recommendation or guiding treatment decisions for the patient depending on whether the patient's cancer is luminal A or luminal B subtype. Suitably, the patient's breast cancer has been identified, e.g. previously identified, as being hormone receptor positive and Her 2 negative.

According to a variation of the third aspect of the invention there is method for providing a treatment recommendation or guiding treatment decisions for a patient with hormone receptor positive Her 2 negative breast cancer, comprising determining the expression levels of a panel of biomarkers, applying these to an algorithm capable of identifying whether the patient is likely to have a luminal A or luminal B cancer and making a treatment recommendation or guiding treatment decisions based thereon, wherein the panel of biomarkers comprises (i) at least one of: KIF23, AURKA, MCM2, CCNA2, PCNA or MCM4; or (ii) MKI67 and at least one of the following: KIF23, AURKA, MCM2, CCNA2, PCNA or MCM4; or (iii) MKI67, KIF23, CCNA2 and PCNA.

According to a fourth aspect of the invention there is provided a method of treating a patient with hormone receptor positive Her2 negative breast cancer, comprising determining whether the patient has luminal A or luminal B breast cancer according to the method of the first aspect of the invention, wherein if the patient's cancer is classified as luminal A they are treated with (or administered) a regime that includes administration of one or more bisphosphonates and/or endocrine therapy, and if the patient's cancer is classified as luminal B they are treated with a regime that includes administration of one or more bisphosphonates, endocrine therapy and/or chemotherapy.

Suitably, the chemotherapy comprises administration of one or more drugs selected from the group consisting of: an anthracycline, e.g. doxorubicin hydrochloride or epirubicin hydrochloride; a taxane, e.g. paclitaxel or docetaxel; a vinca alkaloid, e.g. eribulin mesylate or vinblastine; an antimetabolite, e.g. capecitabine, fluorouracil, gemcitabine hydrochloride or methotrexate; a platinum-based agent, e.g. carboplatin or cisplatin; an epothilone, e.g. ixabepilone; cyclophosphamide, a Cdk4/6 inhibitor, e.g. abemaciclib, palbociclib or ribociclib; a poly-ADP-ribose polymerase (PARP) inhibitor, e.g. olaparib or talazoparib; a PI3K inhibitor, e.g. alpesilib; a PD-L1 inhibitor, e.g. atezolizumab or pembrolizumab; an aurora kinase inhibitor, e.g. alisertib; an ubiquitin proteasome inhibitor; a BCL2-inhibitor; an mTOR inhibitor, e.g. everolimus; an aromatase inhibitor, e.g. letrozole, anastrozole, exemestane; tamoxifen; and, a bisphosphonate agent, e.g. zoledronic acid or sodium clodronate.

According to a variant of the fourth aspect of the invention there is provided a drug selected from an anthracycline, e.g. doxorubicin hydrochloride or epirubicin hydrochloride; a taxane, e.g. paclitaxel or docetaxel; a vinca alkaloid, e.g. eribulin mesylate or vinblastine; an antimetabolite, e.g. capecitabine, fluorouracil, gemcitabine hydrochloride or methotrexate; a platinum-based agent, e.g. carboplatin or cisplatin; an epothilone, e.g. ixabepilone; cyclophosphamide, a Cdk4/6 inhibitor, e.g. abemaciclib, palbociclib or ribociclib; a poly-ADP-ribose polymerase (PARP) inhibitor, e.g. olaparib or talazoparib; a PI3K inhibitor, e.g. alpesilib; a PD-L1 inhibitor, e.g. atezolizumab or pembrolizumab; an aurora kinase inhibitor, e.g. alisertib; an ubiquitin proteasome inhibitor; a BCL2-inhibitor; an mTOR inhibitor, e.g. everolimus; a bisphosphonate agent, e.g. zoledronic acid or sodium clodronate, for use in a method of treating luminal B breast cancer, wherein the method comprises determining whether patient cancer has luminal A or luminal B subtype breast cancer according to the method of the first aspect of the invention, and if the patient has luminal B breast cancer, administering an effective amount of the drug to the patient.

According to a variant of the fourth aspect of the invention there is provided a drug selected from: tamoxifen, raloxifene, fulvestrant, toremifene, goserelin, leuprolide, triptorelin, anastrozole, exemestane, and letrozole, palbociclib, ribociclib, and abemaciclib for use in a method of treating luminal A breast cancer, wherein the method comprises determining whether patient cancer has luminal A or luminal B subtype breast cancer according to the method of the first aspect of the invention, and if the patient has luminal A breast cancer, administering an effective amount of the drug to the patient

As will be appreciated by the person of skill in the art, such drug is likely to be in a pharmaceutical composition that comprises the active ingredient and one or more pharmaceutically acceptable excipients and be in a form and dose suitable for administration to a subject.

The term “pharmaceutically-acceptable excipient” as used herein means one or more compatible solid or liquid fillers, diluents or encapsulating substances that are suitable for administration into a human. The term “excipient” denotes an organic or inorganic ingredient, natural or synthetic, with which the active ingredient is combined to facilitate the application. Types of suitable excipient are salts, buffering agents, wetting agents, emulsifiers, preservatives, compatible carriers, diluents, carriers, vehicles, supplementary immune potentiating agents such as adjuvants and cytokines that are well known in the art and are available from commercial sources for use in pharmaceutical preparations (see, e.g. Remington: The Science and Practice of Pharmacy with Facts and Comparisons: Drugfacts Plus, 20th Ed. Mack Publishing; Kibbe et al., (2000) Handbook of Pharmaceutical Excipients, 3rd Ed., Pharmaceutical Press; and Ansel et al., (2004) Pharmaceutical Dosage Forms and Drug Delivery Systems, 7th Ed., Lippencott Williams and Wilkins). Optionally, the pharmaceutical compositions contain one or more other therapeutic agents or compounds. Suitable pharmaceutically acceptable excipients are relatively inert and can facilitate, for example, stabilisation, administration, processing or delivery of the active compound/agent into preparations that are optimised for delivery to the body, and preferably directly to the site of action.

The pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

When administered, the drug is administered in pharmaceutically acceptable preparations/compositions.

Administration may be topical, i.e., substance is applied directly where its action is desired, enteral or oral, i.e., substance is given via the digestive tract, parenteral, i.e., substance is given by other routes than the digestive tract such as by injection. Large biologic molecules are typically administered by injection.

Pharmaceutical compositions for parenteral administration (e.g. by injection), include aqueous or non-aqueous, isotonic, pyrogen-free, sterile liquids (e.g. solutions, suspensions), in which the active ingredient is dissolved, suspended, or otherwise provided (e.g. in a liposome or other microparticulate). Such liquids may additionally contain one or more pharmaceutically acceptable carriers, such as anti-oxidants, buffers, stabilisers, preservatives, suspending agents, and solutes that render the formulation isotonic with the blood (or other relevant bodily fluid) of the intended patient. In particular embodiments, the composition may be lyophilised to provide a powdered form that is ready for reconstitution as and when needed. When reconstituted from lyophilised powder the aqueous liquid may be further diluted prior to administration. For example, diluted into an infusion bag containing 0.9% sodium chloride injection, USP, or equivalent, to achieve the desired dose for administration. In particular embodiments, such administration can be via intravenous infusion using an intravenous (IV) apparatus.

Suitably, the drug (e.g. active agent) is formulated in accordance with routine procedures as pharmaceutical compositions adapted for intravenous administration to human beings. Typically, the active agent for IV administration is in solution, e.g. in sterile isotonic aqueous buffer. Where necessary, the compositions can also include a solubilizing agent. Compositions for IV administration can optionally include a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule. Where the active compound is to be administered by infusion, it can be dispensed, for example, with an infusion bottle containing sterile pharmaceutical grade water or saline. Where the active compound is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavouring agents such as peppermint, oil of wintergreen, or cherry; colouring agents; and preserving agents, to provide a pharmaceutically palatable preparation. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like.

Compositions for use in accordance with the present invention can be formulated in conventional manner using one or more physiologically acceptable excipients. Thus, the active agent and optionally another therapeutic or prophylactic agent and their physiologically acceptable salts and solvates can be formulated into pharmaceutical compositions for administration by inhalation or insufflation (either through the mouth or the nose) or oral, parenteral or mucosal (such as buccal, vaginal, rectal, sublingual) administration. In one aspect, local or systemic parenteral administration is used.

For oral administration, the compositions can take the form of, for example, tablets or capsules prepared by conventional means with pharmaceutically acceptable excipients such as binding agents (e.g., pre-gelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavouring, colouring and sweetening agents as appropriate.

The pharmaceutical compositions for use the treatment methods of the invention are for administration in an effective amount. An “effective amount” is the amount of a composition that alone, or together with further doses, produces the desired response.

Suitably, the drug can be administered as a pharmaceutical composition in which the pharmaceutical composition comprises between 0.1-1 mg, 1-10 mg, 10-50 mg, 50-100 mg, 100-500 mg, or 500 mg to 5 g of the active agent.

Many of the drugs listed above are already commercially available. The acquisition or preparation of a suitable pharmaceutical composition of the drug and the dosage to administer to a subject is within the capabilities of a person of skill in the art.

### Kits

The materials for use in the methods of the present invention are ideally suited for preparation of kits produced in accordance with well-known procedures. The invention thus provides kits comprising agents for the detection of expression of the disclosed biomarker. Such a kit may comprise separate containers, each with one or more of the various reagents (typically in concentrated form) utilized in the methods, e.g., the kit may contain in separate containers one or more oligonucleotides or antibodies (either already bound to a solid matrix or packaged separately with reagents for binding them to the matrix), control formulations (positive and/or negative), and/or a detectable label, as well as other reagents such as buffers, nucleotide triphosphates, reverse transcriptase, DNA polymerase, RNA polymerase packaged together in the form of a kit. Instructions (e.g., written, or on electronic medium, e.g. CD-ROM, etc.) for carrying out the assay may be included in the kit. The oligonucleotides may be primers suitable for PCR amplification of a biomarker derived amplification product or hybridisation probes capable of hybridising to and thus detecting the presence of an amplification product.

Thus, the present invention also provides kits comprising one or more reagents suitable for determining the expression levels of the biomarkers measured in the methods of the invention described herein.

According to fifth aspect of the invention there is provided a kit of parts comprising a set of oligonucleotide primer pairs wherein each primer pair is capable of selectively hybridising to one of the transcripts in a panel of genes and creating a PCR amplification product, and instructions for use, wherein the panel of genes comprises:
at least 1 gene selected from the group consisting of: KIF23, CCNA2, AURKA, MCM2, MCM4 and PCNA; or
at least 2 genes selected from the group consisting of: KIF23, CCNA2, MKI67, AURKA, MCM2, MCM4 and PCNA, optionally wherein one of the at least two genes is MKI67; or
MKI67, KIF23, CCNA2 and PCNA.

By primer pair we mean the two primers which together amplify up an amplification product by PCR or other amplification method.

Optionally, the kit also comprises at least one probe capable of selectively hybridising to each amplification product. Such probe may be termed a “companion probe” to the primers used to generate the amplification product, i.e. it is used to detect the presence of the particular amplification product generated when using the primer pair.

Each primer pair and companion probe can therefore be used to determine the presence of and expression level of a particular biomarker. They can therefore be referred to as a primer pair/companion probe combination.

Optionally, each primer pair and/or companion probe in the kit is housed in the same container Optionally, each primer, primer pair and/or companion probe in the kit is housed in a separate container.

Optionally, the kit also comprises at least one set of primers capable of selectively hybridising to one of the transcripts selected from the group consisting of: ESR1, PGR, ERBB2 and keratin 5, and creating a PCR amplification product, and optionally at least one probe capable of selectively hybridising to the amplification product. Optionally the probe is labelled, such as with a fluorescent label, to aid detection.

Optionally, the kit also comprises a pair of amplification primers and an amplification product detection probe (companion probe) for a reference gene (e.g. control gene).

Thus, optionally the kit also comprises one or more primer pairs capable of selectively hybridising to and creating a PCR amplification product from one or more control gene transcripts.

Optionally, the kit also comprises instructions for use. Suitably, the kit also comprises means for interpreting the expression data, such means could be a software package capable of weighting the expression levels of each detected biomarker and providing a call (such as: yes/no, high/low proliferation score, or luminal A/luminal B).

Suitably, the kit also comprises a software package that includes a calling algorithm capable of using the expression levels of the biomarkers into an output call, such as whether the test sample comprises cells indicative of luminal A or luminal B (Her2 negative) breast cancer or cells with high or low proliferation.

In particular embodiments, the kit of parts described herein can be used for determining (i) the proliferation status of the cancer cells, or (ii) whether a breast cancer is luminal A or luminal B status of a breast cancer sample.

According to a sixth aspect of the invention there is provided a kit of parts comprising:
(i) at least one binding moiety capable of specifically binding to a biomarker protein selected from: KIF23, CCNA2, AURKA, MCM2, MCM4 and PCNA; or
(ii) at least two distinct binding moieties each capable of specifically binding to a different biomarker protein selected from: KIF23, CCNA2, MKI67, AURKA, MCM2,
MCM4 and PCNA, optionally wherein one of the at least two biomarker proteins is MIK67; or
(iii) at least four distinct binding moieties each capable of specifically binding to a different biomarker protein selected from: MKI67, KIF23, CCNA2 and PCNA.

Suitably the binding moiety is an antibody or antibody fragment capable of selectively binding to the biomarker. Optionally, the binding moiety is labelled, such as fluorescently labelled. Optionally, the kit also comprises a binding moiety for a reference protein. Optionally, the kit also comprises instructions for use. Suitably, the kit also comprises means for interpreting the expression data, such means could be a software package capable of weighting the expression levels of each detected biomarker and providing a call (such as: yes/no, high/low proliferation score, or luminal A/luminal B).

According to a seventh aspect the invention there is provided a biomarker selected from: KIF23, AURKA, MCM2, CCNA2, PCNA and MCM4 for use as a breast cancer subtyping marker or as a marker of relapse free survival.

The following Examples and Figures serve to illustrate the invention. These Examples and Figures are in no way intended to limit the scope of the invention, but rather as examples from which equivalents will be recognized by those of ordinary skill in the art.

Unless it is apparent from the context, each of the embodiments listed above can be applied for use in any of the aspects of the invention.

### DESCRIPTION OF THE FIGURES

FIG. 1. Plots for CCNA2 for A) single marker expression between luminal A and B HER2 negative cases, B+C) ROC curves calculated without and combined with MKI67 expression, and D) overall survival by expression.

FIG. 2. Top Performing Combination of Markers from N=1 to N=7 as determined via the logistic regression (LR) and support vector classifier (SVC) models. MKI67 is shown for reference.

FIG. 3. Average Performance metrics (LR+SVC) for the top 10 performing combinations of markers for the discrimination of luminal A v luminal B (HER2 negative) cases.

### EXAMPLES

### Example 1: In Silico Target Selection

### Samples and Clinical Data

Patient samples and clinical data were extracted from the METABRIC database (Rueda et al., Nature 567 (7748): 399-404, 2019; Curtis et al., Nature 18; 486 (7403): 346-52, 2012). RNA expression levels are reported as microarray probe expression values, with accompanying clinical annotations. The dataset was screened to remove samples without probe expression data, and to remove samples which had non-concordant clinical annotation between different iterations of the Metabric database. Screening was also carried out to remove probes that recognised multiple genes as well as those with the lowest 30% mean expression across samples. Full reconstruction of breast cancer subtypes for each sample from biomarker data was not possible since MKI67 data was not available for this dataset. Thus, luminal A and B status was defined using the Pam50 Subtype (Picornell et al., BMC Genomics 3; 20 (1): 452, 2019) detailed within the dataset. To remove luminal B HER2 positive samples from the analysis, cases were removed by filtering for positive HER2 IHC result.

After filtering 944 patients were identified, comprising of 767 luminal A and 177 luminal B (HER2 negative) samples. After filtering for probe sets that matched more than 1 target gene and probes with the lowest 30% mean expression across samples 21792 probes were available for analysis.

### Candidate Target Selection

To identify probes displaying significant differences in expression between luminal A and B (HER2 negative) samples, three statistical tests were used. An Anova, t-test (standard and Welch's) both one- and two-sided, and a Kolmogorov-Smirnov test. For each analysis, the results were ranked by p-value and the top 200 significantly expressed probes selected. For each probe a count of occurrences across the five analyses was carried out. The top 200 probes that occurred in the most analyses were taken forward for further consideration.

Since the analysis was carried out on a probe basis rather than target basis these 200 probes corresponded to a total of 174 unique target genes that discriminate between luminal A and B (HER2 negative) samples in this dataset.

To further narrow the candidate list, we used a logistic regression model to calculate the mean performance for each probe to differentiate luminal A from B (HER2 negative) samples. Over 100 iterations samples were randomly assigned to a training (80%) or testing (20%) set. The outputs of each model (accuracy, precision, and sensitivity) were used to plot a receiver operator curve (ROC) for each target. A similar analysis was carried out for each single probe expression combined with the expression value for MKI67. These ROC were used to generate AUC (area under the curve) values for probe set comparison. Target gene expression levels and relapse free survival over 10 years for each target was also assessed to aid in final target choice using cox regression.

To recreate the subtypes in the Metabric dataset, the PAM50 derived subtype associated with each case as part of the dataset's clinical information was used. For the luminal spectrum of breast tumours, a concordance of 81%-85% has been reported between PAM50-based luminal A tumours and the IHC-based surrogate subtype, whereas 35%-52% of luminal B tumours are also classified as IHC luminal A (Bernard et al., J Clin Oncol 27 (8): 1160-7, 2009).

We examined the subtype classification in regards MKI67 expression levels within the dataset, where luminal A tumours should possess low expression, and luminal B high, since proliferation as determined by MKI67 is the differentiating factor between these subtypes. IHC for MKI67 shows only a modest correlation with RNA levels (Sinn et al., BMC Cancer 17:124, 2017), however for the extremes of expression, the lowest and highest expression levels should correlate well within the luminal A and B subtype classification. It was apparent in the data however, that 30% of the samples within the 90-100% percentile of MKI67 expression were characterised as luminal A rather than luminal B using PAM50 typing, agreeing with published mis-classification frequencies. To control for this misclassification within the data a secondary analysis between the lowest 0-10th and the highest 90-100th percentiles of MKI67 was carried out using the criteria of a 1.1-fold change between sample group means, with multiple testing controlled for using 50 permutations of the false discovery rate. Hierarchical clustering was carried out using the clustering function on dChip. 234 significant genes passed these criteria.

Comparison of the top 200 probe list and MKI67 expression filtering resulted in a list of 84 common genes. Data was combined from all analyses (single marker, single marker+MKI67 expression, and low v high KI67 expression) and the results in each analysis ranked by ROC performance and the top 20 targets considered for inclusion into our panel of proliferative markers.

To select the final markers, we also considered the phase during the cell cycle at which the target gene was expressed. MKI67 expression is highest in the G2/M phase of the cell cycle, as such to capture more information on cell proliferation, markers that were expressed in alternate phases of the cell cycle were sought out. Cell cycle information was determined for the top 20 targets using Cyclebase (https://cyclebase.org/). Metabric provides expression data in a normalised format-no information on the abundance of target genes can be gained from this dataset. As such we also considered the expression level of each target gene in breast cancer tissue and cell lines (https://www.kobic.kr/GEMICCL/). This was carried out to ensure that target genes would be robust to detection using PCR methods. We also considered association of each target with relapse free survival since markers with a possible prognostic component may impart the assay with the ability to determine clinically relevant information.

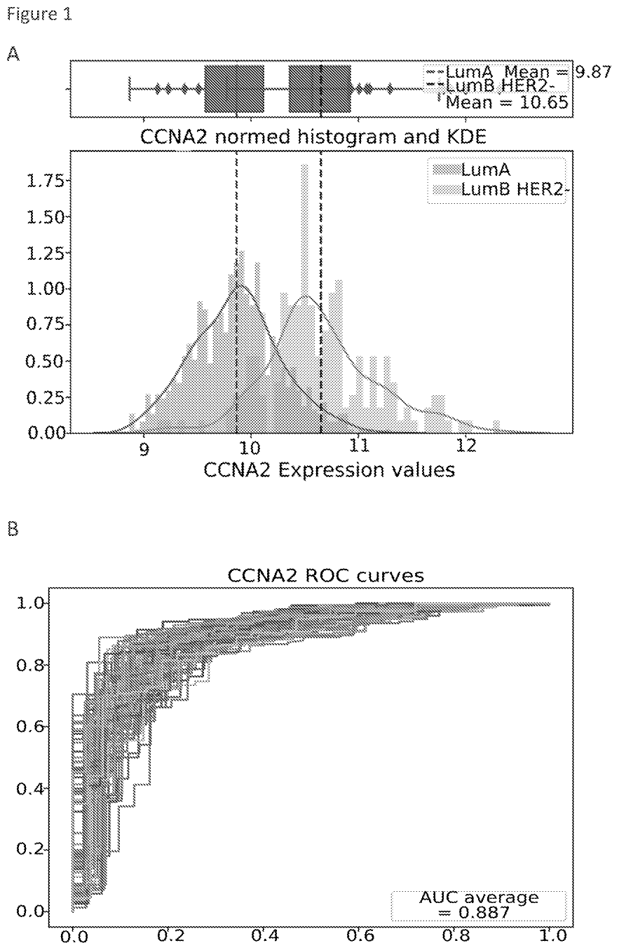

From our final list top 20 targets probes we selected the following targets. CCNA2: the top performing target was associated with an accuracy of 0.804 for luminal A v B (HER2 negative) discrimination. AURKA: was selected over the slightly higher performing AURKB (0.858 v 0.861 for ROC analysis) since two probes corresponding to AURKA were significant in the analysis, increasing the confidence in this target. KIF23: While not at the top of the list of targets for consideration the target has been identified during the prognostic signature search as a marker for relapse free survival in the Metabric cohort. As such the target may confer a prognostic aspect to the subtyping assay and was chosen for further study. An additional 3 targets, PCNA, MCM2 and MCM4 were included as proliferative targets from data supplied by external experts. Of the three only MCM2 appeared in the bioinformatics list of possible targets with a sensitivity of 0.685 to discriminate luminal A from B (Table 3+ FIG. 1).

TABLE 3
Performance Metrics of the top 20 Illumina Probes proliferative targets selected during bioinformatics
study. MKI67 alone is shown for reference. Final selected targets are highlighted in bold.

| | | | | | | | | | | MKI67 | | | | |
|---|---|---|---|---|---|---|---|---|---|---|---|---|---|---|
| | | | | | | | | | | High | | | | |
| | | | | | | | | | | v low | | | | |
| | | | | | | | | | | ex- | | | | |
| | | | | | | | | | | pression | Sur- | Cell cycle expression | | |
| | | Single marker Analysis | | | | Marker + ki67 dual performance | | | | log 2 | vival | Peak | | Expres- |
| | | Accu- | Preci- | Sensi- | ROC | Accu- | Preci- | Sensi- | ROC | fold | P | expres- | | sion |
| Gene | Probe | racy | sion | tivity | curve | racy | sion | tivity | curve | change | value | sion | Other | level* |
| CCNA2 | ILMN_1786125 | 0.804 | 0.957 | 0.794 | 0.887 | 0.776 | 0.932 | 0.781 | 0.816 | 1.11 | 0.229 | G2 | — | CCNA2 |
| AURKA | ILMN_1680955 | 0.790 | 0.939 | 0.793 | 0.858 | 0.746 | 0.917 | 0.757 | 0.813 | 1.15 | 0.818 | M | G2M, M | AURKA |
| AURKA | ILMN_2357438 | 0.786 | 0.937 | 0.790 | 0.857 | 0.757 | 0.920 | 0.768 | 0.812 | 1.15 | 0.210 | M | G2M, M | AURKA |
| FAM64A | ILMN_1728972 | 0.772 | 0.931 | 0.777 | 0.839 | 0.731 | 0.908 | 0.744 | 0.759 | 1.11 | 0.571 | M | — | FAM64A |
| CEP55 | ILMN_1747016 | 0.785 | 0.955 | 0.771 | 0.894 | 0.761 | 0.935 | 0.758 | 0.848 | 1.14 | 0.348 | M | G2M, M | CEP55 |
| AURKB | ILMN_1684217 | 0.775 | 0.942 | 0.771 | 0.861 | 0.758 | 0.919 | 0.770 | 0.816 | 1.15 | 0.462 | G2 | G2M, M | AURKB |
| CKAP2L | ILMN_1751776 | 0.785 | 0.958 | 0.770 | 0.885 | 0.759 | 0.936 | 0.755 | 0.839 | 1.13 | 0.030 | G2 | — | CKAP2L |
| PTTG3P | ILMN_2049021 | 0.771 | 0.940 | 0.767 | 0.872 | 0.738 | 0.923 | 0.739 | 0.822 | 1.15 | 0.983 | G1 | — | PTTG3P |
| BUB1 | ILMN_2202948 | 0.778 | 0.952 | 0.766 | 0.883 | 0.757 | 0.927 | 0.761 | 0.758 | 1.13 | 0.773 | M | G2M, M | BUB1 |
| CDC25C | ILMN_2407619 | 0.775 | 0.952 | 0.762 | 0.863 | 0.707 | 0.909 | 0.711 | 0.755 | 1.1 | 0.579 | G2 | G2 | CDC25C |
| CDCA5 | ILMN_1683450 | 0.769 | 0.947 | 0.758 | 0.858 | 0.737 | 0.918 | 0.743 | 0.815 | 1.19 | 0.374 | G2 | G2/M | CDCA5 |
| PBK | ILMN_1673673 | 0.766 | 0.943 | 0.758 | 0.851 | 0.739 | 0.916 | 0.748 | 0.778 | 1.12 | 0.300 | G2/M | G1/G2 | PBK* |
| PTTG1 | ILMN_1753196 | 0.772 | 0.954 | 0.756 | 0.881 | 0.761 | 0.939 | 0.754 | 0.833 | 1.18 | 0.519 | G1 | G2/M | PTTG1 |
| KIF23 | ILMN_1811472 | 0.767 | 0.949 | 0.754 | 0.870 | 0.725 | 0.919 | 0.727 | 0.787 | 1.11 | 0.320 | G2 | G2/M | KIF23 |
| MCM2 | ILMN_1681503 | 0.696 | 0.920 | 0.685 | 0.780 | 0.666 | 0.894 | 0.669 | 0.713 | 1.13 | 0.301 | M | G1/S, S | MCM2 |
| MKI67 | ILMN_1734827 | 0.701 | 0.919 | 0.693 | — | — | — | — | — | 1.1 | — | M | G2/M | MKI67 |

*Expression level in MCF7 cell line (Luminal A)

### Example 2: Proof of Concept Using RT-qPCR

### Samples and Clinical Information

99 patient samples from resected breast tissue (49 luminal A, 50 luminal B Her2-) and 52 core needle biopsy (CNB) samples (38 luminal A, 14 luminal B Her2-) were used in this study. Samples were obtained as formalin fixed paraffin embedded (FFPE) scrolls 5 μm in 1.5 ml Eppendorf tubes (Pathologie Hamburg-West, Germany).

RNA was extracted from up to 2 FFPE scrolls using the RNeasy DSP FFPE Extraction kit (Qiagen, Germany) according to the manufacturer's protocol, with final elution in 32 μl RNase-free water. Eluted RNA was quantified on a Qubit 4 fluorometer (ThermoFisher Scientific, USA) using a Qubit RNA BR Assay Kit (ThermoFisher Scientific, USA). If insufficient yield to run the RT-qPCR assay, RNA was extracted from additional scrolls, up to a total of 6 scrolls per sample.

### RT-qPCR

### Template

RNA extracted from patient FFPE samples was diluted to 2.5 ng/ul in nuclease free water, total input into RT-qPCR reactions was 10 ng. Nuclease free water was used as a non-template control (NTC).

### Targets

Primers and probes were designed to selectively hybridise to RNA transcript, preferentially designed to encompass the illumina probe sequence corresponding to the Metrabric analysis. Primers were designed across the closest exon/exon boundary to produce RNA specific amplification product. 7 target primer/probe combinations were included in the screening, together with 2 reference genes (IPO8 and PUM1). HPLC-purified primers were supplied by ThermoFisher Scientific, USA, probes were supplied by IDT, USA. Probes were fluorescently-labelled with 5′ 6-FAM™ and a 3′ BHQ1 quencher, with the exception of IPO8 and PUM1 labelled with Tex 615™ and a 3′BHQ2 quencher.

### RT-qPCR

16 ul of template free reagent mix, including target-specific primers (400 nM) and probes (300 nM), Phoenix HotStart Taq DNA polymerase, EnzScript Reverse Transcriptase (RT), and ZipScript buffer (Enzymatics, USA) was added to each well of a 96-well plate. 4 μl of template (negative control, or FFPE-extracted RNA) was added. PCR set-up was performed on the Quant Studio 5 (QS5) (ThermoFisher Scientific, USA). Each sample was screened with 9 master mixes for biomarker targets AURKA, CCNA2, MCM2, MCM4, PCNA, KIF23, MKI67, and the reference genes IPO8 and PUM1. 2 replicates were tested per mix for each sample along with a negative control (NTC) for each mix. Primer sequences were as described in Table 4.

TABLE 4
Primer and probe studies referred to in this study

| | | | Sequence ID | |
|---|---|---|---|---|
| Target | Function | Oligo | Number | Sequence |
| CCNA2 | Target | Forward | SEQ ID NO: 1 | TGCTGTTAGCCTCAAAGTTTGAAG |
| | | Primer | | |
| | | Reverse | SEQ ID NO: 2 | CTTGGTGTAGGTATCATCTGTAATG |
| | | Primer | | |
| | | Probe | SEQ ID NO: 3 | TATACCCCCCAGAAGTAGCAGAGTTTGTG |
| PCNA | Target | Forward | SEQ ID NO: 4 | CCTGAACTTCTTTACAAAAGCCA |
| | | Primer | | |
| | | Reverse | SEQ ID NO: 5 | CTCTACAACAAGGGGTACATCT |
| | | Primer | | |
| | | Probe | SEQ ID NO: 6 | CTCTTCAACGGTGACACTCAGTATGT |
| KIF23 | Target | Forward | SEQ ID NO: 7 | ATCATTGCGGCAGGTTACTC |
| | | Primer | | |
| | | Reverse | SEQ ID NO: 8 | GCAGAGCGTGATCGTCTG |
| | | Primer | | |
| | | Probe | SEQ ID NO: 9 | CAACCTGATCAGAACGCACCACCAA |
| MCM2 | Target | Forward | SEQ ID NO: 10 | CGCTTCAAGAACTTCCTGCG |
| | | Primer | | |
| | | Reverse | SEQ ID NO: 11 | CTCACGGTTCTCTTTGCACAT |
| | | Primer | | |
| | | Probe | SEQ ID NO: 12 | CCACAACGTCTTCAAGGAGCGCATC |
| MCM4 | Target | Forward | SEQ ID NO: 13 | AAGGACTACATTGCCTACGC |
| | | Primer | | |
| | | Reverse | SEQ ID NO: 14 | CATGTCTACATAAGCCTCGATGA |
| | | Primer | | |
| | | Probe | SEQ ID NO: 15 | C+GGCTAAGTGAGGAAGCCAGC |
| AURKA | Target | Forward | SEQ ID NO: 16 | CCACCTTCGGCATCCTAATATTCT |
| | | Primer | | |
| | | Reverse | SEQ ID NO: 17 | TGTTCCAAGTGGTGCATATTCCA |
| | | Primer | | |
| | | Probe | SEQ ID NO: 18 | TTC+CATGATGCTACCAG+AGTCTACC |
| MKI67 | Target | Forward | SEQ ID NO: 19 | GCTTGTTTGGAAGGGGTATTGA |
| | | Primer | | |
| | | Reverse | SEQ ID NO: 20 | CTGCTCATGGATTTCAATTTTGC |
| | | Primer | | |
| | | Probe | SEQ ID NO: 21 | AGCTTCCTGTTGTGTCAAAACAAC |
| IPO8 | Ref | Forward | SEQ ID NO: 22 | CATCATTCTTCAGTGCAAAGG |
| | Gene | Primer | | |
| | | Reverse | SEQ ID NO: 23 | CTCTCCAAAACAAGTTGAACG |
| | | Primer | | |
| | | Probe | SEQ ID NO: 24 | AGGGGAATTGATCAGTGCATTCCACTCT |
| PUM1 | Ref | Forward | SEQ ID NO: 25 | CATCCTTGGGATTCGGAAGTAG |
| | Gene | Primer | | |
| | | Reverse | SEQ ID NO: 26 | CAGTGTTGGAGTTTGCAACTG |
| | | Primer | | |
| | | Probe | SEQ ID NO: 27 | CCCTTGGAGGGTTTGGAACA |

+in the sequence refers to a locked nucleic acid base - (LNA) and refers to o modification of the nucleotide backbone

Cycling conditions were: 50° C. for 10 minutes; 95° C. for 30 seconds; and 40 cycles of 94° C. for 10 seconds and 60° C. for 30 seconds. Fluorescence intensity from the hydrolysed probes was measured in the FAM or Tex 615 channel during the 60° C. Annealing-Extension step in each cycle.

QS5 runs were analysed applying the software automatic baseline and the following threshold setting for each target. AURKA-126000, CCNA2-94000, MCM4+MCM2-85000, MKI67-27000, PCNA-90000, IPO8-80000, PUM1-35000 and KIF23-100000.

### Run, Sample and Target Validity Criteria

For the resected samples a total of 41 plates were run for the samples. 5 plates failed run validity due to NTC amplification, all plates were successfully repeated. 5 samples initially failed sample validity due to drop out for one of the reference genes, 4 samples subsequently passed following repeat PCR, one sample was not repeated due to insufficient RNA. 100% of samples eventually passed run and sample validity.

A total of 18 plates were run for the CNB samples. NTC amplification was observed on two plates. A single run failed due to power failure.

### Data Analysis

To adjust for small differences in sample input raw Ct values were processed to calculate delta Ct (mean reference genes-mean target of interest). PCR data from 94 of the resected samples was merged with clinical information, the dataset was well balanced with 47 luminal A and 47 luminal B HER2 samples. PCR data was generated for 44 of the core needle biopsy samples, with 31 luminal A samples and 13 luminal B HER2 negative samples available for analysis.

### Marker Performance

To evaluate each target's ability to discrimination luminal A v luminal B HER2 negative a stratified shuffle split cross validation strategy was applied. Using a logistic model, for each combination of targets, input data was subjected to 100 random 0.7:0.3 test:train splits, and the prevalence of subtypes between test and train data was kept equal. Further, for each data split an independent model was trained using the train data subset and evaluated on the test data subset. Evaluation was done by calculating accuracy, precision, sensitivity and F1 score.

Since there is no “gold-standard” method for binary classification problems (Luminal A v B falls into this category), performance results from two models were compared. The first a logistic regression model (LM) and the second a support vector classifier (SVC model). Both model implementations were imported from the scikit-learn V0.22.1, Python module for machine learning. The F1 score (the harmonic mean of the precision and sensitivity values) was averaged across the model results, and the averaged F1 was used for target combination ranking. The highest possible value of an F-score is 1, which indicates perfect precision and sensitivity. The F1 score penalises models with a poor precision-sensitivity trade-off and thus is better for model comparison than using a single parameter.

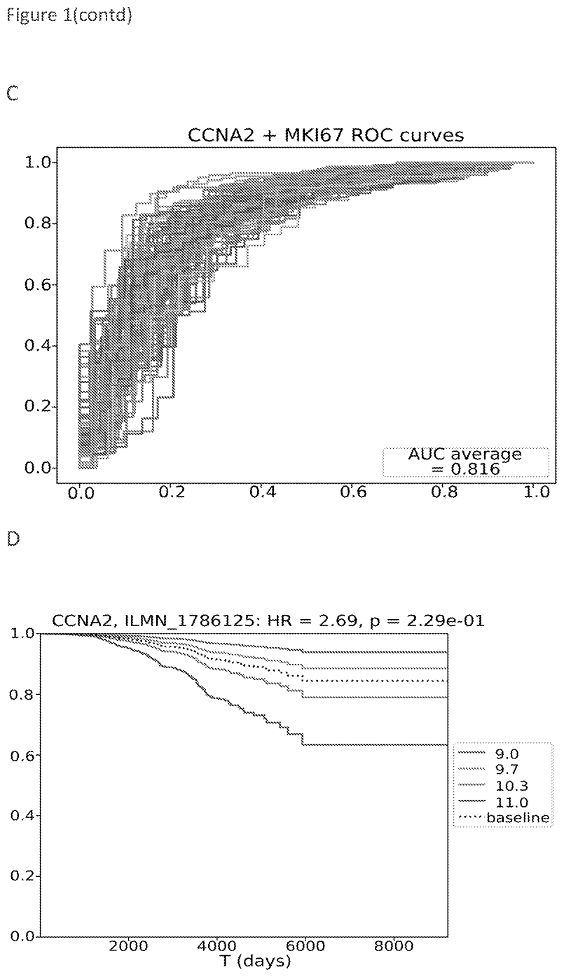

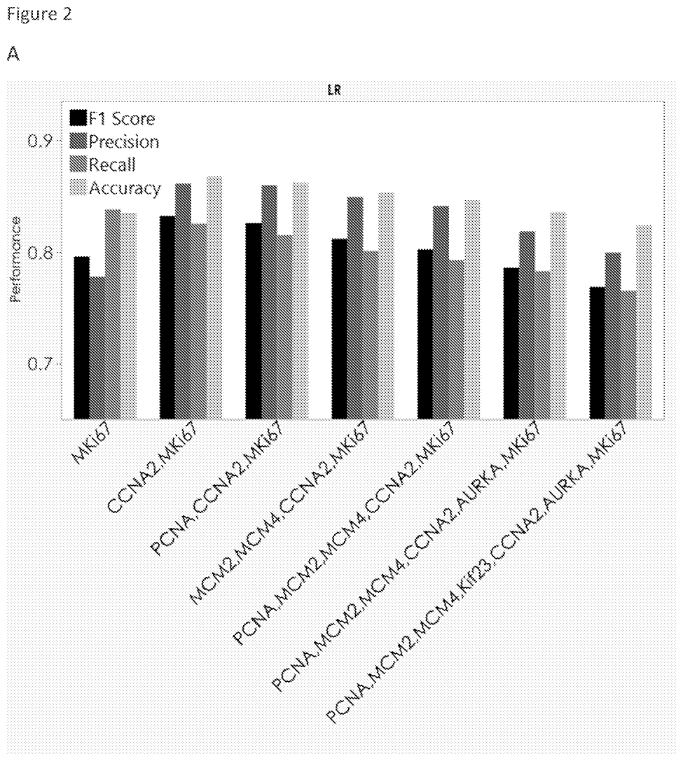

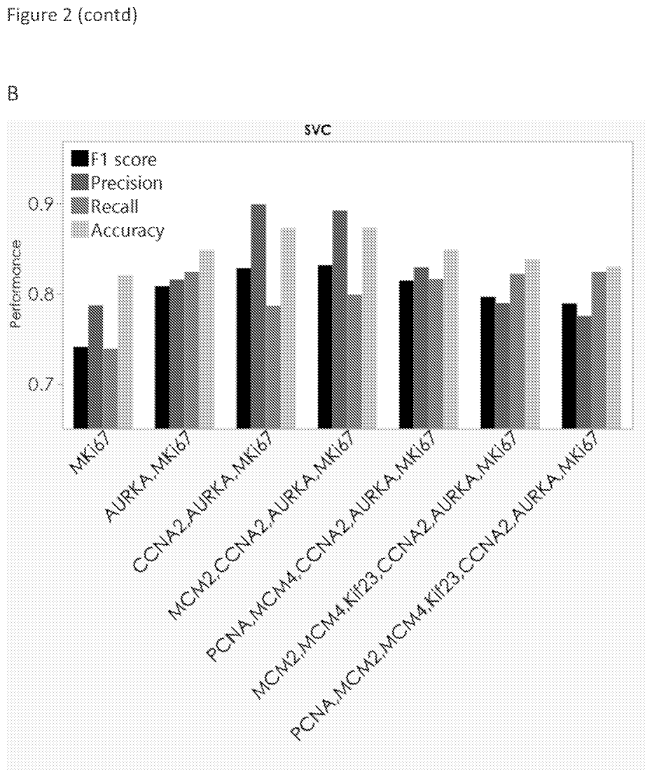

All the possible combinations from any one marker to all 7 combined (N=127) to discriminate luminal A v luminal B (HER2) were evaluated using both the logistic and SVC model, the top performing combinations from each model are plotted in FIG. 2.

The results of the analyses clearly show that adding up to three additional markers to the model increases the performance of the model to discrimination luminal A and luminal B (HER2 negative) samples. However, as the number of markers increases the performance of the model to discriminate the cases drops. The SVC model performs well including up to 4 markers in the analysis (0.741 for MKI67 F1 score compared to a maximal score of 0.832). The LR model shows a maximum increase in F1 score at 2 markers (0.796 for MKI67 alone, v 0.8325). However the LR model maintains performance for 3 markers (maximum F1 score 0.826). As such a maximum of 4 markers was decided on for the assay.

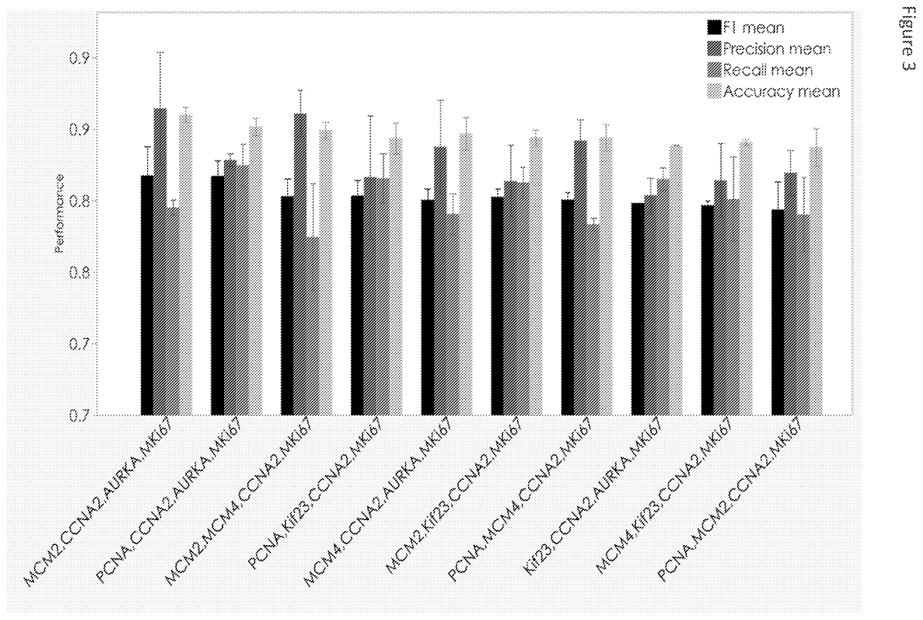

The average F1 score for the LR and SVC analyses was calculated, and the performance metrics plotted for the combinations of 4 markers. As can be seen in FIG. 3, similar performance is seen for many different marker combinations the top 10 combinations here differ by only 0.024.

1. A method for classifying a patient's cancer comprising determining the expression level of at least one biomarker selected from: KIF23, AURKA, MCM2, CCNA2, PCNA and MCM4, and classifying the cancer based on the expression levels of the biomarker detected.

2. The method of claim 1, wherein the expression level of the at least one biomarker determines the proliferation status of the cancer.

3. The method of claim 1, wherein at least two biomarkers selected from: MKI67, KIF23, AURKA, MCM2, CCNA2, PCNA and MCM4, are measured, optionally wherein one of the at least two biomarkers is MKI67.

4. The method of claim 1, wherein the cancer is breast cancer.

5. The method of claim 4, wherein the patient's breast cancer is classified as luminal A or luminal B subtype.

6. The method according to claim 5, wherein the patient's cancer is hormone receptor positive and her2 negative.

7. The method according to claim 5, wherein the expression levels detected are compared to or correlated with reference values characteristic of luminal A and luminal B breast cancer subtypes.

8. The method according to claim 1, wherein the expression level of at least 3, 4, 5, 6, or all 7, biomarkers are determined.

9. The method according to claim 4, wherein the expression level of the or each biomarker is weighted to determine the cancer proliferation status and/or classify the cancer as being a luminal A or luminal B subtype.

10. A method for prognosing or predicting clinical outcome for a patient diagnosed with cancer, comprising determining the expression level of at least one biomarker selected from: KIF23, AURKA, MCM2, CCNA2, PCNA and MCM4, and prognosing or predicting the clinical outcome for the cancer patient from these expression values.

11. The method according to claim 10, wherein at least two biomarkers selected from: MKI67, KIF23, AURKA, MCM2, CCNA2, PCNA and MCM4, are measured, optionally wherein one of the at least two biomarkers is MIK67.

12. (canceled)

13. The method according to claim 10, wherein the cancer is breast cancer.

14-15. (canceled)

16. The method according to claim 1, wherein the biomarker expression level is determined based on the amount of the RNA transcript level detected.

17. (canceled)

18. The method according to claim 1, wherein the expression level for each measured biomarker is determined quantitatively.

19-20. (canceled)

21. The method according to claim 1, wherein the assessment is carried out on a cancer sample previously isolated from the patient, optionally as a solid or liquid biopsy sample or during surgery.

22. The method according to claim 21, wherein the cancer sample is fresh, frozen, or paraffin-embedded and fixed.

23. The method according to claim 1, wherein the biomarkers evaluated comprises:
(i) at least four biomarkers selected from: KIF23, CCNA2, MKI67, AURKA, MCM2, MCM4 and PCNA; or
(ii) MKI67 and at least three biomarkers selected from KIF23, CCNA2, AURKA, MCM2, MCM4 and PCNA; or
(iii) MKI67, KIF23, CCNA2 and PCNA.

24-33. (canceled)

34. A kit of parts comprising a set of oligonucleotide primer pairs wherein each primer pair is capable of selectively hybridising to one of the transcripts in a panel of genes and creating a PCR amplification product, and instructions for use, wherein the panel of genes comprises at least 2 genes selected from the group consisting of: KIF23, CCNA2, MKI67, AURKA, MCM2, MCM4 and PCNA.

35. A kit of parts according to claim 34, comprising a set of oligonucleotide primer pairs wherein each primer pair is capable of selectively hybridising to one of the transcripts in a panel of genes and creating a PCR amplification product, and instructions for use, wherein the panel of genes comprises: genes selected from the group consisting of: MKI67, KIF23, CCNA2 and PCNA, wherein the kit also comprises at least one probe capable of selectively hybridising to each amplification product.

36-39. (canceled)

40. The kit of parts according to claim 34, for use in determining (i) the proliferation status of the cancer cells, or (ii) whether a breast cancer is luminal A or luminal B status of a breast cancer sample.

41. (canceled)

42. The method according to claim 10, wherein the biomarker expression level is determined based on the amount of the RNA transcript level detected.

43. The method according to claim 10, wherein the biomarkers evaluated comprise:
(i) at least four biomarkers selected from: KIF23, CCNA2, MKI67, AURKA, MCM2, MCM4 and PCNA;
(ii) MKI67 and at least three biomarkers selected from KIF23, CCNA2, AURKA, MCM2, MCM4 and PCNA; or
(iii) MKI67, KIF23, CCNA2 and PCNA.
